(19) United States
(12) Patent Application Publication
(10) Pub. No.: US 2023/0301250 A1
(43) Pub. Date: Sep. 28, 2023

(54) SYSTEM AND METHOD FOR HARVESTING FUNGAL BIOMASS FROM PLANAR BED

(71) Applicant: Kismet Labs, Inc., Middletown, DE (US)

(72) Inventors: Isabella Iglesias-Musachio, Berlin (DE); Quentin Loth, Berlin (DE); Emma Giancaterino, New York, NY (US); Rafael Philippini, Guaratingueta (BR)

(73) Assignee: Kismet Labs, Inc., Middletown, DE (US)

(21) Appl. No.: 18/114,067

(22) Filed: Feb. 24, 2023

Related U.S. Application Data
(60) Provisional application No. 63/313,335, filed on Feb. 24, 2022.

(51) Int. Cl. A01G 18/70; A01G 18/80

(52) U.S. Cl. CPC A01G 18/70; A01G 18/80

(57) ABSTRACT

Harvesting methods and systems for harvesting fungal biomass from a growth container using one or more meshes is described. The growth container includes a first mesh through which the fungal biomass grows. A second mesh can be disposed over the first mesh such that the fungal biomass grows further through the second mesh. A rotator or a slider can be coupled to the second mesh and configured to move the second mesh relative to the first mesh causing the fungal biomass to shear and transport the fungal biomass on the second mesh to a delivery area.

### CROSS REFERENCE TO RELATED APPLICATIONS

The present application claims the benefit of U.S. Patent Application No. 63/313,335, filed on Feb. 24, 2022 the subject matter of which is incorporated herein by reference in its entirety.

### BACKGROUND

Fungal biomass such as mycelium can be cultivated as an ingredient for meat substitutes. Replacing animal meat by a fungal-based alternative has the potential to dramatically reduce the impact that animal farming has on the climate, environment, and on animal welfare, while providing a new option for meat eaters that can naturally mimic the texture, taste, and look of meat. While mycelium has great potential as a meat-substitute due to its natural fibrous texture and nutritional profile, the low production yield, manual labor-intensive harvesting and quality issues associated with its cultivation cost prevents this ingredient from reaching the unit economics needed to match customer's price expectations (i.e. to compete with the cost of industrially produced animal meat).

### BRIEF DESCRIPTION OF THE DRAWINGS

Various embodiments in accordance with the present disclosure will be described with reference to the drawings, in which:

FIG. 1 illustrates an example growth system, in accordance with various embodiments.

FIG. 1A is an isometric view of a growth box, in accordance with various embodiments.

FIG. 1B is a section view of the growth box of FIG. 1A, in accordance with various embodiments.

FIG. 1C is a side view of the growth box of FIG. 1A with an internal tubing system, according to an example embodiment.

FIG. 1D is a side view of the growth box of FIG. 1A with internal tubing system designed for heat management, according to an example embodiment.

FIG. 2A is an isometric view of a growth cylinder according to various embodiments.

FIG. 2B is a section view of the growth cylinder of FIG. 2A.

FIG. 2C is a side view of the growth cylinder of FIG. 2A with internal tubing system, according to an example embodiment.

FIG. 2D is a side view of the growth cylinder of FIG. 2A with internal tubing system designed for heat management, according to an example embodiment.

FIG. 2E is a side view of the growth cylinder of FIG. 2A with auger screw mechanism, according to an example embodiment.

FIG. 2F illustrates a solid-liquid substrate system, according to some embodiments.

FIG. 3A illustrates a growth cylinder having two longitudinal sides configured for growing fungal biomass (e.g., mycelium), in accordance with some embodiments.

FIG. 3B is a top view of an array of growth cylinders of FIG. 3A disposed within a growth chamber.

FIG. 3C is a side view of the array of growth cylinders of FIG. 3B disposed within a growth chamber.

FIG. 4A illustrates a growth cylinder coupled to a rotation system, in accordance with some embodiments.

FIG. 4B illustrates another growth cylinder coupled to the rotation system, in accordance with some embodiments.

FIG. 5 illustrates a side view of a growth cylinder with a mechanized cutter that moves vertically alongside the growth cylinder for harvesting fungal biomass (e.g., mycelium).

FIG. 6A illustrates a side view of a growth cylinder with a mechanized cutter that is brought into close contact with and kept stationary while the growth cylinder is rotated for harvesting fungal biomass (e.g., mycelium).

FIG. 6B illustrates a side view of a growth cylinder with a mechanized cutter that rotates about the fungal biomass (e.g., mycelium) for harvesting.

FIG. 7 illustrates a mechanized cutter with an adjustable height that separates the fungal biomass (e.g., mycelium) from the substrate material.

FIG. 8A illustrates a peeling method for harvesting fungal biomass (e.g., mycelium) with a flexible mesh.

FIG. 8B illustrates a roller method for harvesting fungal biomass (e.g., mycelium) from flexible mesh.

FIG. 9A illustrates a method for harvesting fungal biomass (e.g., mycelium) from a mesh using a cutter.

FIG. 9B illustrates the harvesting of the fungal biomass (e.g., mycelium) from a rotating growth cylinder with a cutter.

FIG. 10 illustrates a double mesh system for growing and harvesting fungal biomass (e.g., mycelium) from the growth container.

FIG. 11 graphs the exposed surface in cm² and percent biological efficiency of different growth containers described herein compared to existing systems.

FIG. 12A illustrates sectional views of a planar bed harvesting system with (i) a first mesh positioning, and (ii) another way of the first mesh positioning, in accordance with some embodiments.

FIG. 12B illustrates moving a mesh for harvesting from the planar bed system of FIG. 12A in accordance with some embodiments.

FIG. 12C illustrates a platform of the planar bed-based harvesting system in accordance with some embodiments.

FIG. 12D illustrates the planar bed-based harvesting system using a cutter in accordance with some embodiments.

FIG. 12E illustrates an enlarged view of harvesting portion of the system of FIG. 12D.

FIG. 13 is a flow chart of an example method for growing fungal biomass, in accordance with various embodiments.

FIG. 14 is a flow chart of an example method for harvesting fungal biomass, in accordance with various embodiments.

FIG. 15 is a flow chart of another example method for harvesting fungal biomass, in accordance with various embodiments.

### SUMMARY

The present disclosure provides reliable and automatable processes for the growth and harvesting of fungal biomass e.g., mycelial biomass, fruiting body, or other fungi, including for use in the food industry. Growth systems for fungal biomass provided herein can increase the amount and quality of yield, and facilitate automation and scaling for mass production of fungal biomass. Also, harvesting systems provided herein can enable fast, efficient and cleaner harvesting of the fungal biomass, with improved fungal biomass quality. The harvesting systems provided herein can result in little to no post-processing of the harvested fungal biomass, since the fungal biomass does not need to be cleaned of any remaining substrate, leading to lower down-stream processing costs.

According to an aspect, a growth system for growing fungal biomass (e.g., mycelium) can include a growth container with one or more meshes through which fungal biomass can grow. The growth system may include a growth container having a surface area (e.g., about 400 cm² or more) exposed to a growth chamber environment. The growth container is configured to hold an inoculated substrate, where the growth container is a cylinder. The growth system can also include a first mesh or sections of a first mesh at least partially extending along a circumference of the growth container. The first mesh is configured to expose the inoculated substrate to the growth chamber environment and allow growth of fungal biomass through the first mesh.

In various embodiments, the growth container has a first longitudinal growth side and a second longitudinal growth side opposite to the first longitudinal growth side. The growth container is constructed of a material selected from stainless steel, aluminum, polypropylene, polyethylene, nylon, polycarbonate, silicone, biodegradable material, or biobased material. The first mesh is constructed of a material selected from stainless steel, aluminum, polypropylene, polyethylene, nylon, polycarbonate, silicone, biodegradable material, or biobased material. The first mesh stretches along on a bottom surface, and the growth container is hung in the growth chamber.

In various embodiments, the growth system can further include a flexible and removable second mesh outermost to said first mesh on said growing container. The first mesh and/or a second mesh have an aperture, which are the same or vary in a range of 0.5 mm to 25 mm. The spacing for the apertures or openings of the first mesh and/or the second mesh are in a range of 1 mm to 50 mm from center to center.

In some embodiments, the growth system can further include a rotator system configured to rotate or shift orientation of the growth container during incubation. In some embodiments, the growth system further includes a tubing system extending inside the growth containers configured to maintain moisture content of the substrate in a range between 50% to 70%. In an embodiment, the tubing system is used as an irrigation and drainage system, allowing a continuous or intermittent feeding of water and/or nutrients to the substrate. In an embodiment, the tubing system allows a continuous or intermittent flow of a liquid to cool the growth container during incubation. In an embodiment, the tubing system is used as a delivery system to allow feeding of a liquid inoculum to the substrate pre-incubation.

In various embodiments, the growth system can further include a growth chamber. The growth chamber can include controlled levels of gasses may include carbon-dioxide, oxygen, and/or nitrogen; controlled temperature; controlled air flow; and/or controlled relative humidity. Growth system where the growth chamber environment may include from about 0.04% to about 10% CO₂, a temperature of from about 15 C to about 35 C, and a relative humidity from about 80% to about 100%. An air flow is activatable and pilotable inside the growth chamber. In an embodiment, the growth chamber is a hermetic chamber, and where the growth chamber enables total darkness. In an embodiment, the growth chamber provides a controllable or programmable lighting system of different wavelengths.

According to another aspect, a growth system includes a growth container configured to hold a substrate for growing the fungal biomass. The growth container can include: one or more solid frame portions, where at least one solid frame portion extending at a bottom of the growth container; and a first side mesh and a second side mesh opposite to the first side mesh. The first side mesh and the second side mesh being coupled to the one or more solid frame portion. The first side mesh and the second side mesh are substantially planar and extend in respective vertical planes. The first mesh and the second mesh are configured to expose the substrate to a growth chamber environment and allow growth of the fungal biomass along a direction normal to the top of the substrate and through the first side mesh and the second side mesh.

According to yet another aspect, a growth system includes a matrix of growth containers. The matrix of growth containers include a first set of vertically distributed growth containers and a second set of vertically distributed growth containers spaced horizontally from the first set of vertically distributed growth containers. Each growth container has a surface area exposed to a growth chamber environment and configured to hold an inoculated substrate. Each growth container is a cylinder or a rectangular box. The growth system can further include one or more meshes coupled to the growth containers, where each mesh is vertically oriented and at least partially extends along a circumference of each growth container. Each mesh is configured to expose the inoculated substrate to the growth chamber environment and allow growth of fungal biomass through the mesh.

In various embodiments, the first set of vertically distributed growth containers and the second set of vertically distributed growth containers are in an in-line configuration, or a staggered configuration. The in-line configuration can include: at least one growth container of the first set of vertically distributed growth containers is horizontally aligned with at least one growth container of the second set of vertically distributed growth containers. The staggered configuration can include each growth container of the first set of vertically distributed growth containers being offset from each one growth container of the second set of vertically distributed growth containers.

In various embodiments, the growth system can further include a hanging system configured to support the first set of vertically distributed growth containers and the second set of vertically distributed growth containers. The hanging system may include a set of cables connectable to each growth container of the first set of vertically distributed growth containers and the second set of vertically distributed growth containers.

In various embodiments, the growth system can further include a rotator configured to rotate, via the cable, each growth container of the first set of vertically distributed growth containers, and the second set of vertically distributed growth containers.

According to yet another aspect, a method for growing fungal biomass includes providing a growth system. The growth system includes a growth container holding an inoculated substrate, and a mesh extending along a circumference of the growth container and configured to expose the inoculated substrate to a growth chamber environment and allow growth of fungal biomass through the mesh. The growth container is a vertically oriented cylinder or a rectangular box allowing growth of the fungal biomass along a vertically oriented longitudinal side of the cylinder or the rectangular box. Further, the method can include incubating the growth container in the growth chamber under conditions suitable for the growth of fungal biomass.

In some embodiments, the method can further include feeding, via a tubing system, water or liquid (e.g., liquid nutrients), inside the growth container during incubation. In some embodiments, the method can further include passing a coolant or heat through a tubing system inside the growth container during incubation. In some embodiments, the method can further include moving, rotationally or linearly, the growth container during the incubation.

According to yet another aspect, a harvesting system may include a growth container holding a substrate. The growth container being disposed vertically within a growth chamber environment. The growth container being coupled to a mesh around a circumference of the growth container such that fungal biomass is grown from the substrate and through the mesh.

In various embodiments, the harvesting system can further include a cutter configured to move relative to the growth container to harvest the fungal biomass across the mesh while preventing the substrate from sticking to the harvested fungal biomass. In some embodiments, the cutter is a sliding cutter, where the sliding cutter is slidable along a vertical direction. In some embodiments, the cutter is configured to move circumferentially around the growth container to harvest the fungal biomass from the mesh. In some embodiments, the cutter is stationary and the growth container is configured to rotate about a central axis to harvest the fungal biomass from the mesh. In various embodiments, the fungal biomass is grown on a first longitudinal side and a second longitudinal side of the growth container, and the cutter is slidable along the first longitudinal side of the growth container to harvest the fungal biomass. In some embodiments, the growth container is configured to rotate about a central axis to align the second longitudinal side with the cutter to slidably harvest the fungal biomass from the second longitudinal side. In some embodiments, the growth container is stationary while the cutter slides along the first or the second longitudinal sides of the growth container.

In some embodiments, the harvesting system can include a matrix of growth containers. The matrix of growth containers include a first set of vertically distributed growth containers and a second set of vertically distributed growth containers having a horizontal spacing therebetween. In some embodiments, the cutter is disposed within the horizontal spacing and movable horizontally to harvest the fungal biomass from at least one growth container of the first set of vertically distributed growth containers and at least one growth container of the second set of vertically distributed growth containers. In some embodiments, the horizontal spacing is greater than twice the horizontal length of the fungal biomass extending from the mesh of the respective growth container of the matrix of growth containers.

According to yet another aspect, a method for harvesting fungal biomass includes providing a growth system, incubating the growth container, aligning a cutter, and harvesting the fungal biomass. The growth container holds an inoculated substrate, and a mesh extends along a circumference of the growth container and is configured to expose the inoculated substrate to a growth chamber environment and allow growth of fungal biomass through the mesh. The growth container is a vertically oriented cylinder or a rectangular box allowing growth of fungal biomass along a longitudinal side of the cylinder or the rectangular box. The method can further include incubating the growth container in the growth chamber under conditions suitable for growing the fungal biomass. The method can further include aligning a cutter along the mesh of the growth container. The method can further include harvesting the fungal biomass from the growth container by relatively moving the cutter along the mesh of the growth container.

In various embodiments, the harvesting includes sliding the cutter along a longitudinal side of the growth container. In various embodiments, the harvesting includes pulling sections of the growth container through a fixed cutter separating the fungal biomass from the mesh and the substrate. In some embodiments, the harvesting can include rotating the growth container along its central axis with the cutter placed against the mesh to separate the fungal biomass from the growth container. In various embodiments, a second mesh surrounds the mesh at the circumference of the growth container, and the harvesting includes: moving the second mesh from the growth container to cut the fungal biomass from the mesh; and removing the fungal biomass from the second mesh. The harvested fungal biomass contains no portions of the inoculated substrate.

According to yet another aspect, a harvesting system includes a planar bed with a growth container (or bed filled with inoculated substrate) for growing fungal biomass including a first mesh and a removable second mesh. The growth container may include a first mesh through which the fungal biomass grows. A second mesh is disposed over the first mesh such that the fungal biomass grows further through the second mesh. A rotator or a slider is coupled to the second mesh and configured to move the second mesh relative to the first mesh causing the fungal biomass to shear and transport the fungal biomass on the second mesh to a delivery area.

In some embodiments, the rotator is configured to pull the second mesh around and toward the bottom side of the growth container. In some embodiments, the slider is configured to pull the second mesh approximately parallel to the growth container and separated from the growth container. In some embodiments, the second mesh is configured to peel off with respect to the first mesh to separate the fungal biomass from the growth container.

In some embodiments, the harvesting system includes a cutter movably disposed between the first mesh and the second mesh to separate the fungal biomass and the second mesh from the first mesh. In some embodiments, the harvesting system includes a platform located in the delivery area proximate to the growth container so as to receive the fungal biomass from the second mesh; and a stopper located on the platform at an end opposite the growth container so as to stop the fungal biomass from advancing off the platform while forming a fungal biomass layer on the platform for easy handling.

In some embodiments, the harvesting system includes a cutter disposed between the growth container and the platform to separate the fungal biomass on the second mesh while the second mesh is moving. The cutter is an elongated cutter including a cutting edge oriented against the second mesh; and a top surface over which the fungal biomass is movable from the second mesh to the platform.

In some embodiments, the platform can be further coupled to a lift system or a conveyor to convey a harvested fungal biomass layer to a specified location. In some embodiments, the harvesting system further includes a plurality of growth containers and a plurality of harvesting systems. The plurality of growth containers are vertically spaced from each other. Each growth container being the same as the growth container for growing the fungal biomass. A harvesting system includes the second mesh relatively slidable with respect to the first mesh of the respective growth container. The plurality of growth containers are supported on columns. The second mesh is made of plastic, aluminum, or steel. The second mesh is coupled to a motor to automatically harvest the fungal biomass. The second mesh is coupled to a motor to automatically move the second mesh or a manual slider to manually move the second mesh.

According to yet another aspect, a method for harvesting fungal biomass includes providing a growth system for growing fungal biomass, harvesting the fungal biomass using a mesh and transporting the harvested fungal biomass. The growth system includes a growth container holding an inoculated substrate; a first mesh coupled to the growth container and extending over the inoculated substrate such that the fungal biomass grows through the first mesh; and a second mesh disposed over the first mesh such that the fungal biomass grows further through the second mesh. The harvesting involves moving the second mesh relative to the first mesh causing the fungal biomass to shear. Once harvested, the fungal biomass on the second mesh is automatically transported to a delivery area.

In some embodiments, the moving of the second mesh includes rotating a rotator coupled to the second mesh such that the second mesh is pulled over the first mesh and moves the second mesh toward the bottom side of the growth container. The moving of the second mesh includes: pulling the second mesh approximately parallel to the first mesh and the growth container to harvest the fungal biomass over the second mesh; and separating the second mesh from the growth container.

In some embodiments, the transporting of the harvested fungal biomass includes providing a platform in the delivery area proximate to the growth container so as to receive the fungal biomass from the second mesh; and providing a stopper on the platform at an end opposite the growth container so as to stop the fungal biomass from advancing off the platform while forming a fungal biomass layer on the platform for easy handling.

In some embodiments, the method can further include: providing a cutter between the growth container and the platform to separate the fungal biomass on the second mesh while the second mesh is moving. In some embodiments, the transporting of the harvested fungal biomass can include coupling a lift system or a conveyor to the platform; and conveying, via the lift system or the conveyor, the harvested fungal biomass layer to a specified location.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

In various aspects and embodiments, the present disclosure provides fungal biomass (e.g., mycelium, fruiting body, primordia, etc. or a combination) growth systems, harvesting systems, and methods of growing and harvesting the fungal biomass. According to aspects of the present disclosure, a higher yield e.g., of fungal biomass growth can be achieved by exposing multiple surfaces of an inoculated substrate to the environment of an incubator (“growth chamber”), and/or by changing the growth direction of the fungi. These aspects and embodiments result in an improved yield while reducing the amount of substrate and space required to grow fungal biomass, leading to a cost-effective production and more efficient utilization of resources.

Mycelial biomass not grown in liquid fermentors is otherwise conventionally grown in trays. For example, inoculated substrate is packed in a rectangular tray, and placed in a controlled environment incubator, controlling, for example, CO₂ (and optionally other gasses), temperature, air flow, and relative humidity. The mycelial biomass grows from the substrate in an anti-gravitational direction, and only on the one exposed surface of the substrate in the trays. This cultivation operates within a hermetic chamber that enables total darkness. The mycelial biomass will grow in several days on top of the substrate exposed to the environmental conditions inside the growth chamber and create a mat of mycelial biomass that can later be harvested from the substrate with an electric knife, a saw or a cutting wire. These conventional methods of harvesting pure mats of mycelium from a tray system are extremely labor intensive since harvesting is usually done by hand. Furthermore, separating the mycelium from the substrate is complex and leads to product quality degradation. Using these production techniques result in high labor costs and slim unit economics.

This disclosure provides apparatuses and systems for fungal biomass growth and harvesting that can allow for improved growth yields and separation of the fungal biomass from the substrate in a scalable and automatable manner. In various embodiments, the systems and apparatuses described herein can reduce the cost and utilization of resources for growing and harvesting fungal (e.g., mycelial) biomass products, including, but not limited to, for use in the meat alternatives industry.

Various experimental trials were conducted using existing growth systems and a predefined growth chamber environment to evaluate growth of mycelium. However, these studies provided only moderate improvements in growth yield. By using different substrate ingredients in different proportions, further moderate improvements on the growth yield of the tested fungal species could be obtained. To provide more substantial improvements in growth yield, growth containers as discussed herein were used. For example, rectangular and cylindrical containers with meshes on two or more sides were used and subjected to specified growth conditions. These growth containers provided a substantially improved growth yield of mycelium. For example, growth yield improved more than 100%.

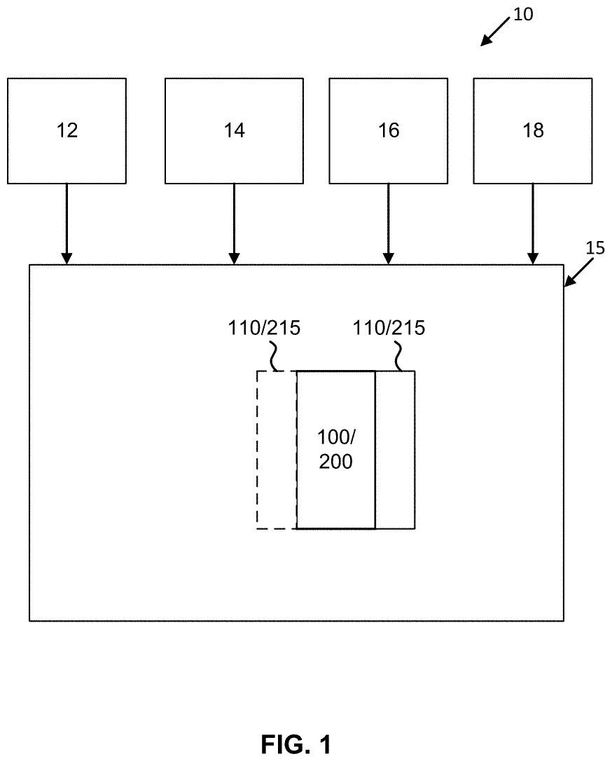

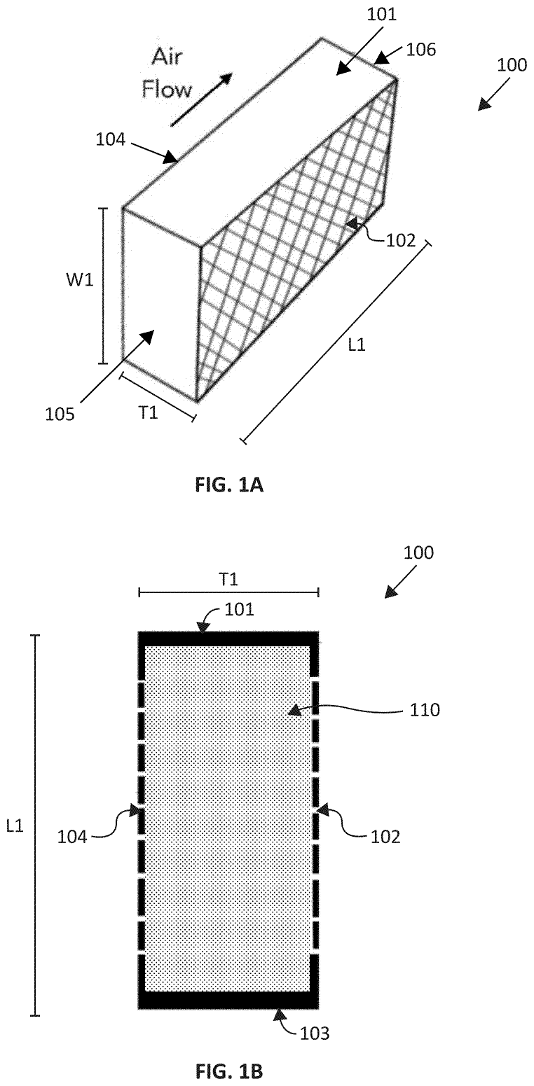

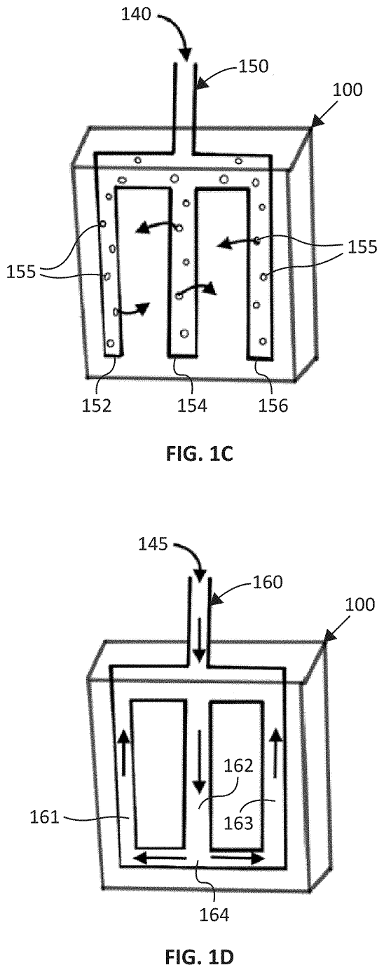

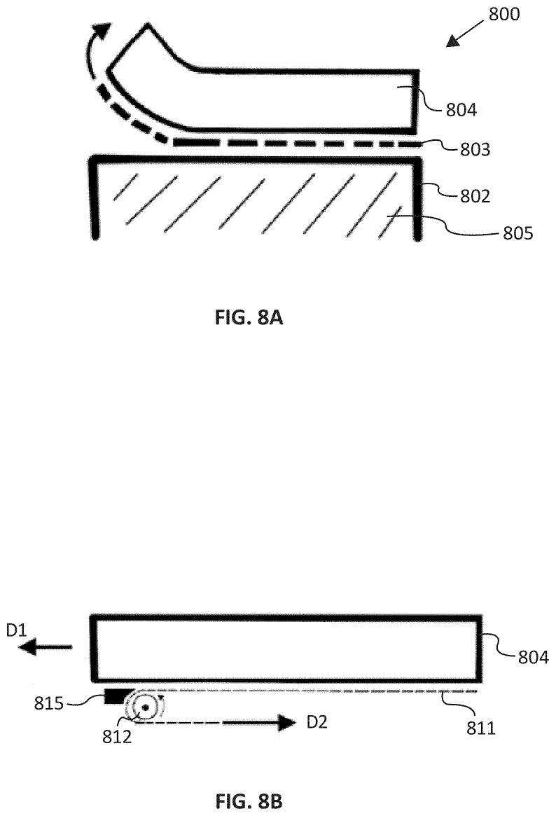

To improve the quality and protection of the harvested mycelial biomass, several additional trials were conducted. First, attempts to separate the grown mycelial biomass from the substrate post-incubation were done using a sharp knife, scissors, a scalpel and an electric knife. All those techniques ended up damaging the mycelial biomass, and parts of the substrate could be found attached to the mycelium, which presents a food safety hazard as well as increased down-stream processing costs. A trial was implemented in the growth chamber with growth system herein packed with inoculated substrate and covered with a metal or plastic sheet pierced with holes, which allowed for mycelium to grow outside the substrate and through the holes of the sheet, and which allowed for the separation of the mycelium from the substrate during harvesting by simply tearing the mycelium from the substrate (e.g., as illustrated in FIG. 8A). The sheet prevented pieces of substrate from staying attached to the mycelium. The size and design of the mesh (e.g., design and pattern of openings and mesh material) can be tuned for optimal growth of fungal biomass, for example, to provide optimal gas exchange between the substrate and the environment of the growth chamber and the moisture within the substrate, while ensuring an easy harvesting process that can protect the integrity and quality of the mycelial biomass. The design of the mesh divider can also vary depending on the selected fungal species, as the fungal biomass can vary in terms of structure, density, and fragility, among other traits depending on the selected fungal species. Examples of growth containers with meshes are discussed herein and illustrated in FIGS. 1B, 2A, 2B, 4A, 4B, 8A-8B, 9A, 9B, 10, and 12A-12C.

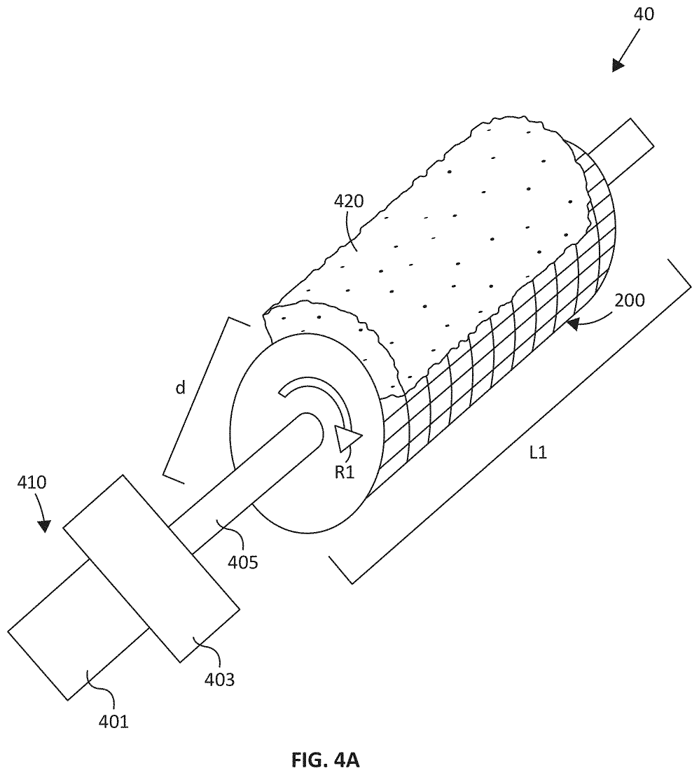

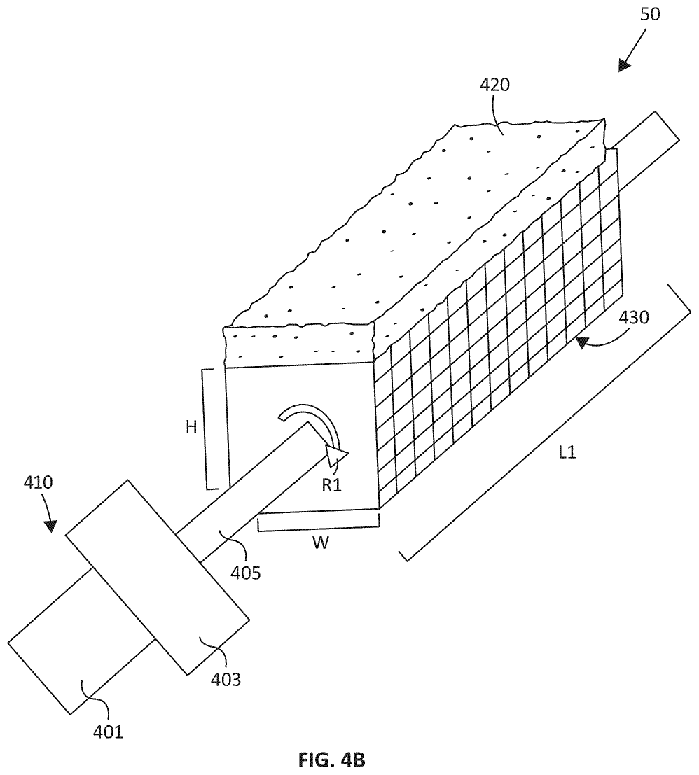

Experimental results disclosed herein demonstrate that by increasing the surface of inoculated substrate in contact with the environment of the growth chamber, we are able to increase the growth rate of the mycelial biomass, in addition to growing biomass in several orientations and surfaces. This increases the mycelium growth yield and allows for a more efficient mycelium harvesting technique. Additionally, the growth system (e.g., including growth container(s)) can be dynamic. For example, growth systems can be adapted to be rotated about one or more axes, i.e., from horizontal to vertical or tilted between different positions (e.g., see FIG. 4A, 4B). In some embodiments, the growth systems can shift between different orientations (e.g., horizontal, vertical, or any other tilted orientation for which the angle may shift during growth). Furthermore, these studies led to a novel design for the substrate holding container that allows for new harvesting methods that can be automated and lead to a scalable industrial process at a controlled cost in comparison to the conventional state-of-the-art techniques.

Accordingly, in one aspect, the disclosure provides a fungal biomass growth system comprising a growth container (e.g., 100, 200, 300, 510. herein) having a surface area exposed to a growth chamber environment of at least about 400 cm². The growth container holds an inoculated substrate for growing a fungal biomass (e.g., mycelium). In various embodiments, the growth container has a surface area exposed to the growth chamber environment of at least about 450 cm², or at least about 500 cm², or at least about 550 cm², or at least about 600 cm², or at least about 650 cm², or at least about 700 cm², or at least about 750 cm², or at least about 800 cm². Existing growth systems or containers provide a surface area for growth of only about 300 cm².

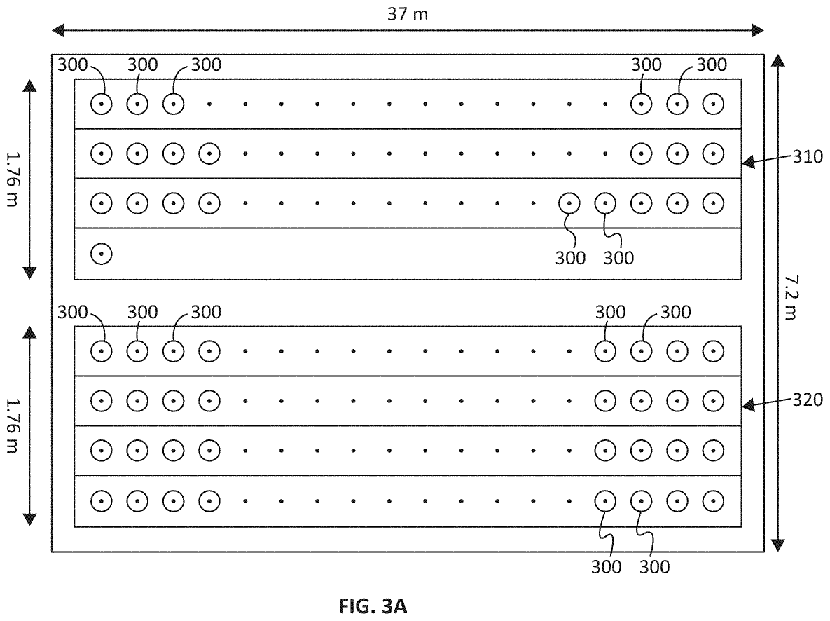

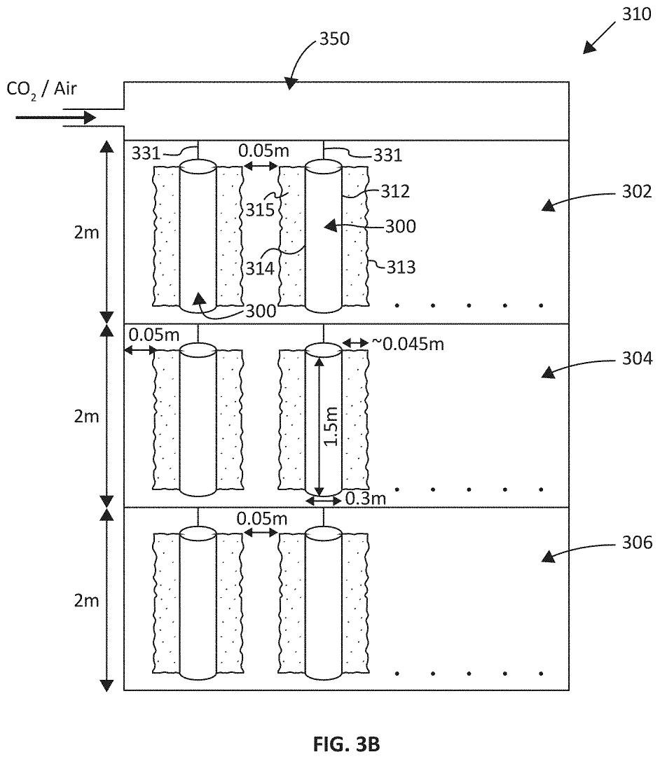

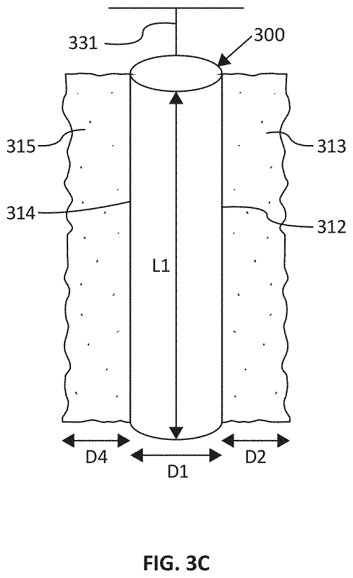

During the cultivation, several growth containers are placed in the growth chamber. For example, in various embodiments (e.g., see FIGS. 3B and 3C), depending on size of the growth chamber, there can be at least 5, or at least 10, or at least 15, or at least 20, or at least 25, or at least 30, or at least 40, or at least 50 of said growth containers in the growth chamber for fungal biomass cultivation. The present disclosure is not limited to a particular number of containers placed within the growth chamber.

The growth containers can take various shapes providing the desired surface area. The growth chamber can adopt cylindrical, elliptical, and spherical shapes. Growth containers can be placed or suspended along the internal space of the growth chambers. Growth containers can be constructed of materials such as stainless steel, aluminum, polypropylene, polyethylene, nylon, polycarbonate, silicone, biodegradable or bio-based material, among others.

In various embodiments, the growth system is configured to be dynamic. Growth container can adopt various shapes, e.g., rectangular, cylindrical, conical, elliptical, spherical, etc., with growth container(s) positioned with multiple orientations adapted to grow fungal biomass on the multiple surfaces by shifting between orientations. In some embodiments, a growth container is rotated or shifted during the growth process, either along a horizontal and/or vertical axis. The dynamic movement exposes each growing surface to nutrients, water, air flow, etc., allowing each surface to cultivate biomass in the appropriate gravitropic direction. In this way, fungal biomass growth can be harvested from all surfaces, rather than being confined to growth on only the surfaces of static growth methods. Exemplary harvesting mechanisms can include a mechanized cutter that moves about the growth container. Alternatively, the harvesting mechanism is brought into close contact with the growth container, and the growth container is moved about the stationary harvesting mechanism. This movement enables the harvesting process to occur vertically (e.g., via the growth cylinder), horizontally (e.g., via the growth box), or by a combination of both while the growth system components shift between positions. In some embodiments, harvesting can occur vertically, for instance via the vertical cylinder to allow for a more efficient harvest process and post-harvest collection.

In some embodiments, the growth container has at least five sides, such as six, seven, or eight sides, and provides for fungal biomass growth (by exposure of the inoculated substrate to the growth chamber environment) on at least two sides, or at least three sides, or at least four sides. In various embodiments, the container is substantially box-shaped (i.e., having six sides that are each substantially rectangular or square). In various embodiments, the growth container provides for fungal growth on three sides, four sides, five sides, or six sides. Other shapes having more than six sides can be employed without departing from the spirit of the invention, including irregular shapes. For example, in some embodiments, growth container dimensions vary in range of height from 15 cm to 300 cm, in length from 15 cm to 300 cm and in width from 3 cm to 80 cm. In some embodiments, the growth container can comprise a mesh structure on sides intended for allowing for fungal growth. In accordance with this disclosure, the mesh may be constructed of a material selected from stainless steel, aluminum, polypropylene, polyethylene, nylon, polycarbonate and silicone. For example, the mesh is placed at least on sides substantially parallel to an air flow within the chamber. In dynamic systems, i.e., upon rotation or shifting along one or more axes, all sides are positioned such that they are at least substantially parallel to air flow within the chamber for some amount of time. These sides can include at least the vertical walls, top, and/or a bottom, while the growth container is placed (e.g., hung or resting on a platform) from any side in the growth chamber (e.g., hung vertically or horizontally), or transitioning between vertical and horizontal states. During the cultivation (i.e., the incubation) the fungal biomass grows around and through the mesh. As described herein, the mesh holds the inoculated substrate in place during cultivation and facilitates harvest of the fungal biomass.

FIG. 1 illustrates an example growth system 10, according to various embodiments. The growth system 10 can include a growth chamber 15 within which one or more growth containers 100 (or 200) and (optionally) related tubing systems (e.g., in FIG. 1C, 1D, 2C, 2D) can be disposed. The growth chamber 15 can include a controlled growth environment wherein one or more growth parameters can be controlled to facilitate growth of specified fungal biomass 110 (or 215) (e.g., mycelium, mushroom, primordia, etc.) on the growth container 100 (or 200). In the illustrated embodiments, environment in the growth chamber 15 can be controlled by controlling one or more growth parameters such as gas, temperature, light, nutrients, gas, moisture, or other growth related parameters. For example, the control system can include, but is not limited to, a gas control subsystem 12, a temperature control subsystem 14, a moisture control subsystem 16, and a lighting control subsystem 18. As an example, the control subsystems 12, 14, 16, and 18 can be configured to provide controlled levels of gases, including but not limited to, carbon-dioxide, oxygen, nitrogen, and/or other gases. The growth chamber may be further provided with controlled temperature, controlled air flow, and/or controlled relative humidity.

As an example, for growing mycelium, growth parameters within the growth chamber 15 can be controlled to include from about 0.04% to about 10% CO₂, a temperature of from about 15° C. to about 35° C., a relative humidity from about 80% to about 100%, or a combination thereof. In some embodiments, air flow can be activated and piloted inside the growth chambers to brush surfaces of the growth container 100 or 200 that expose the inoculated substrate. In some embodiments, the growth chamber 15 can be a hermetic chamber and enable total darkness. In some embodiments, the growth chamber 15 provides a controllable or programmable lighting system of different wavelengths. In some embodiments, a growth chamber may include an array of growth containers (e.g., 3A) to advantageously provide an energy efficient solution. For example, the amount of CO₂ released during incubation can naturally facilitate growth of fungal biomass with minimal or no additional CO₂ pumping energy is expended to control CO₂ levels in the growth chamber.

FIGS. 1A to 1D illustrate examples of a growth container 100, according to various embodiments. The growth container 100 can be a rectangular box with six sides, or other polygonal shaped box having more than six sides. The growth container 100 can be configured to hold a substrate 110 (illustrated in FIG. 1B) for growing mycelium, mushrooms, other appropriate fungal biomass, or a combination thereof. In the illustrated embodiment, the growth container 100 can be an elongated rectangular box, as such alternatively referred to as a growth box 100. For example, the growth box 100 can include six sides—a first side (e.g., a right side), a second side (e.g., a left side), a third side (e.g., a top side), a fourth side (e.g., a bottom side), a fifth side (e.g., a front side), and a sixth side (e.g., a back side). The growth box 100 can include one or more solid frame portions, and two or more meshes coupled to two or more sides (e.g., at least 3, 4, 5, or 6 sides) of the growth box 100. In the illustrated embodiment, the growth box 100 can include a first solid frame portion 101 (e.g., at a top side) and a second solid portion 103 (e.g., at a bottom side). A first side mesh 102 and a second side mesh 104 can be attached to the first and second solid frame portions 101, 103 at opposite sides of the growth box 100. The first side mesh 102 can be planar and extend in a first vertical plane (e.g., the first side of the box 100), and the second side mesh 104 can be planar and extend in a second vertical plane (e.g., the second side of the box 100) parallel to the first vertical plane.

In some embodiments, the growth box 100 can be vertically oriented such that the meshes 102, 104 are on the sides (e.g., left side and the right side) and the solid frame portions 101, 103 can be on the top side and the bottom side, respectively. Accordingly, the substrate 110 can stay in the growth box 100 while exposing its meshed sides to specified growth environments. The growth box 100 can facilitate growth of a fungal biomass such as mycelium, mushroom, or a combination thereof to grow a hybrid biomass e.g., in horizontal direction.

The growth box 100 can be made of e.g., stainless-steel, plastic, wood, or other appropriate material. In some embodiments, the box 100 can be substantially smaller in one of three dimensions (e.g., length L1, width W1, and thickness T1). For example, the box can be L1 long and W1 wide to provide large growth surfaces (e.g., at meshes 102, 104), and T1 thin along the surface facing an air flow. The mesh 102, 104 can be suitable for the substrate 110 to be filled and held within the growth box 100. The mesh can be however fine and permeable enough so that it allows for gas exchange and moisture control between the substrate 110 and a growing chamber environment (e.g., outside the box 100, see FIG. 1). The pattern of mesh openings and spacings allow for fungal biomass (e.g., mycelium, mushroom, primordia, or a combination thereof) to grow around and through it. For example, sizes of the mesh openings may be in the range of 0.5 mm to 35 mm.

The growth box 100 can be placed vertically (e.g., see FIGS. 1B and 3C) or horizontally (e.g., see FIG. 3C) inside a growth chamber, and allow the fungal biomass to grow on a plurality of sides (e.g., 102, 104), as opposed to in an anti-gravitational direction. Alternatively or additionally, the growth box 100 can be configured to transition between vertical and horizontal positions. Allowing the fungal biomass to grow on a plurality of sides (e.g., 102, 104) of the growth box 100 can facilitate increased (e.g., maximizes) the growth yield and lead to better use of the space available in the growth chamber compared to conventional methods. In addition to the improved surface for fungal biomass growth, the growth box 100 can be intermittently fed liquid or gasses through a tube placed inside the growth box 100.

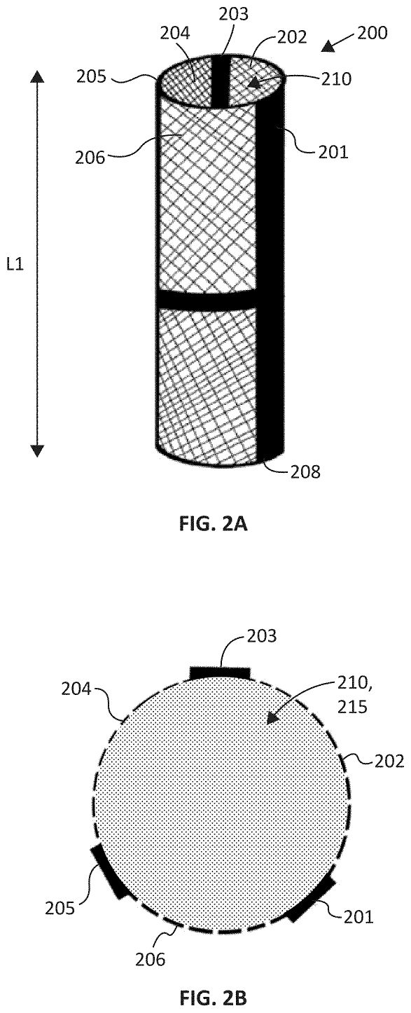

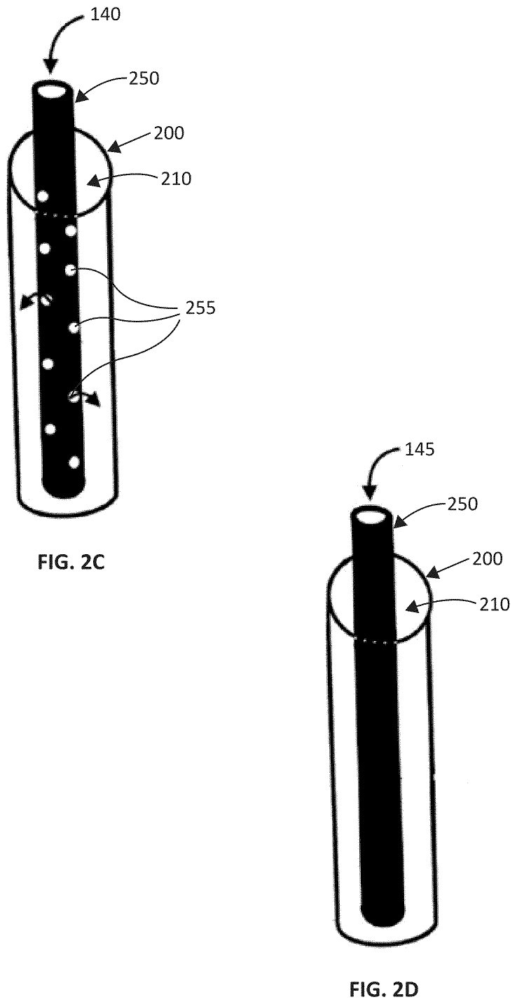

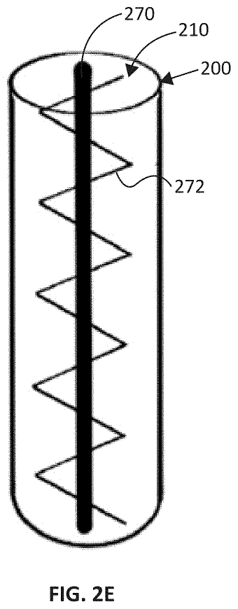

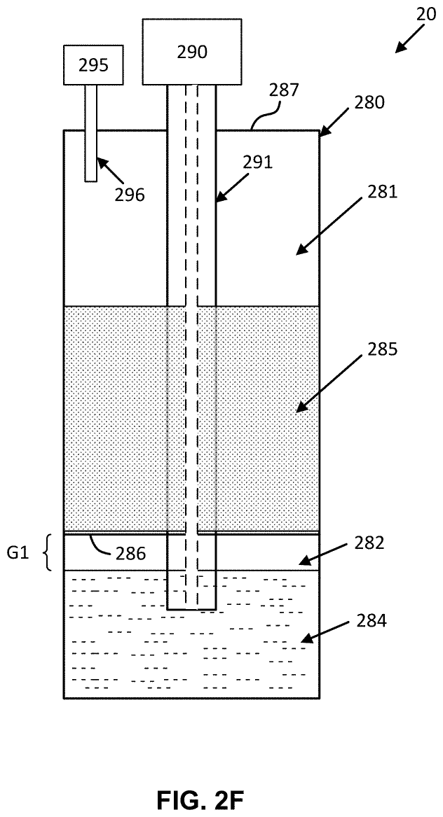

According to various embodiments, the growth containers (e.g., 100 in FIG. 1 or 200 in FIG. 2A) herein may comprise a permanent or removable tubing system (e.g., 150, 250) for irrigation, supplementation, drainage, inoculation or cooling inside the growth containers. The tubing system can connect to a liquid (e.g., liquid nutrients), gas, or liquid inoculum source during cultivation. The tubing system allows for a continuous or intermittent feeding of liquid or gas to the substrate during incubation. In some other embodiments, the tubing system allows for a continuous or intermittent flow of cool water inside the growth container.

The tubing system can take the shape of one or multiple tubes inside the growth container, connected or independent. Liquids (e.g., liquid nutrients), gas or liquid inoculum can be delivered to the substrate during incubation. An internal tubing system passing through the substrate can allow a flow of liquid to cool the substrate during incubation. For example, tubing systems configured for liquid or gas flow to a substrate are illustrated in FIGS. 1C and 2C.

FIG. 1C illustrates an example tubing or flow system 150, according to an embodiment. The flow system can be fed through the growth chamber or growth container(s), for example, under the substrate 110 (in FIG. 1B) and along several surfaces, such that liquid and/or gas flow 140 can be controlled to pass through each surface as it can be rotated/translated to a specific position. For example, the tubing system 150 can include a first tube 152, a second tube 154, and a third tube 156. Each of the tubes 152, 154, 156 can include holes 155 sized and spaced to release water or gas to the substrate 110. For example, the tubes 152 and 156 can be used to deliver liquid or gas close to outer portions of the substrate 110 and the tube 154 can be centrally disposed to deliver liquid or gas at a center of the substrate 110.

In some embodiments, the tubing systems 150, 250 can be used to irrigate the substrate 110 (shown in FIG. 1B) with water and/or provide a cooling mechanism. High moisture is essential for adequate mycelial growth, adding water to the substrate 110 during the incubation phase allows for a higher yield of mycelium. Additionally, this tube can also be used to inoculate the substrate with a liquid inoculum before incubation, or to feed nutrients to the substrate, or gasses (such as CO₂) during incubation.

As an example, the liquid inoculum can comprise a nutrient-rich media containing a specific microorganism or a group of specific microorganisms. The microorganisms, which were previously isolated and grown in solid (agar plates, agar slants, paper strips) or liquid phases (pre-inoculum), are transferred to flasks or bioreactors containing the sterile liquid culture media. The mixture of cells and nutrients grows in pre-established conditions such as running time, temperature and medium composition, which may vary according to the selected microorganism.

The inoculum can be prepared using different culture media compositions and the nutrients may be incorporated individually or in combination to the solvent. In various embodiments, nutrients include carbohydrates, amino acids, and fatty acids. The media preparation can be selected according to its composition and divided as chemically defined or complex. The chemically defined medium preparation can usually be elaborated with water as a solvent, while complex medium presents an undefined amount of components that can be used as nutrients for the fungal development. The complex medium can be obtained from the cooking process of many sugar-rich sources such as, but not limited to, barley, corn, rice and rye. Sugar sources can also be obtained from beetroot and sugarcane, as well as its respective molasses. Agro-industrial side streams can also be used for complex medium elaboration and the nutrients recovery can require physical (extrusion, hydrodynamic cavitation, milling, microwave, ultrasonication, etc.) chemical (acids, alkaline, ionic liquids, organosolv, etc.) or biological (cellulolytic and hemicellulolytic enzymes) pretreatments. These processes can be used alone or in combination, increasing the accessibility of sugars and consequently the efficiency in sugar recovery during the pretreatment process.

The carbohydrates can be monomeric sugars, such as pentoses (xylose, arabinose), hexoses (glucose, fructose, galactose, mannose), dimers (cellobiose, maltose, sucrose), oligomers and polysaccharides (cellulose, pectin and starch). Carbohydrates can be also outsourced from the sugar industry or obtained from agricultural feedstock sources. Agricultural feedstock can be obtained from different cultivars, using many residual byproducts as materials such as, but not limited to bagasses, bran, leaves, oily cakes, peels, roots, stems and stalks. The nitrogen source can be obtained from different bran types, which are produced as side streams of determined grain processing. Inorganic sources of nitrogen can be ammonium nitrate, sodium nitrate, ammonium chloride, ammonium sulphate, ammonium dihydrogen phosphate, brewer's spent yeast. They can also be obtained from sources such as peptone and yeast extract. The fatty acids can be obtained from different vegetable oils, such as, but not limited to canola, corn, cotton, olive, sesame, soybean, etc. Other adjuvants can be implemented in medium elaboration, such as micronutrients (phosphates, sulfates, and salts) additives (corn steep liquor, surfactants, vignasse, etc.) and vitamins which can be incorporated for increasing the nutritional value and the overall cell production during inoculum preparation.

In some embodiments, a tubing system within the growth container can have a cooling function, using water, oil or air as a medium. During incubation, heat can be produced in the substrate through metabolic activity. In order to preserve the integrity of the microorganism and therefore the yield of fungal biomass produced, actively cooling the substrate can be beneficial and increase fungal biomass growth. For example, tubing systems configured for cooling or heating a substrate are illustrated in FIGS. 1D and 2D.

FIG. 1D illustrates another example tubing system 160 configured to deliver heat to the substrate (e.g., 110). In the illustrated embodiment, the tubing system 160 can be fed through the growth chamber or growth container(s), for example, under the substrate 110 (in FIG. 1B) and along several surfaces, such that heat or coolant 145 can be controlled to pass through each surface as it can be rotated/translated to a specific position. For example, the tubing system 160 can include a first tube 161, a second tube 162, a third tube 163, and a fourth tube 164 connecting each of the tubes 161-163. The tubes 161-163 may be parallel to each other and the fourth tube 164 can be orthogonally oriented to connect the tubes 161-163. The second tube 162 may be referred to as a central tube 162 and the fourth tube 164 may be referred as a connecting tube 164. The central tube 162 can receive a heat or coolant 145 and direct the heat or coolant 145 through the connecting tube 164 into the side tubes 161 and 163. The heat or coolant 145 can flow in one direction (e.g., downward) through the central tube 162 and an opposite direction (e.g., upward) through the side tubes 161 and 163. Accordingly, the substrate can be evenly and uniformly heated or cooled.

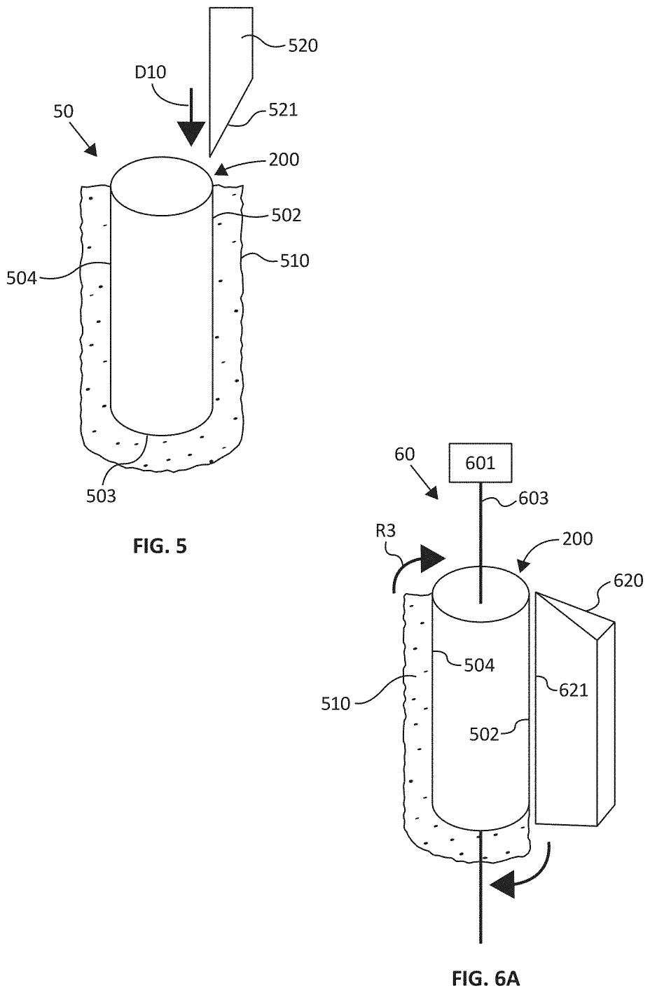

FIGS. 2A to 2D illustrate another example of a growth container 200 according to various embodiments. In illustrated embodiments, the growth container 200 can be a cylinder or can be substantially cylindrical, as such can be referred as a cylindrical growth container. However, the present disclosure is not limited to rectangular or cylindrical shapes, and other container shapes such as conical, elliptical, or spherical are possible. In the illustrated embodiments of FIGS. 2A and 2B, the growth container 200 may include a mesh or sections of mesh 202, 204, 206 for exposing an inoculated substrate (e.g., 215 within an hollow interior 210) to a growth chamber environment. The meshes 202, 204, 206 can be attached by frames 201, 203, and 205 and arranged to form a cylindrical shape. The meshes 202, 204, 206 can extend along a longitudinal length L1 of the growth container 200. As an example, dimensions of the cylindrical growth container 200 can vary in a range of height (e.g., the longitudinal length L1) from 15 cm to 300 cm and diameter from 5 cm to 80 cm. The cylindrical growth container 200 may have meshes (e.g., 202, 204, and 206) on at least about 50% or at least about 75% of the circumferential surface of the cylindrical growth container 200. In some embodiments, the growth container 200 may optionally a bottom mesh 208 (e.g., see also 503 in FIG. 5) on a bottom surface of the growth container 200 to facilitate fungal biomass growth (e.g., see FIG. 5) through the bottom mesh 208.

In various embodiments, the growth container 200 or portions of the growth container 200 as well as the mesh (e.g., 202, 204, 206) can be constructed of stainless steel. In some embodiments, the growth container 200 and the mesh (e.g., 202, 204, 206) can be constructed of other materials conducive for fungal growth and harvest according to embodiments described herein. Such materials include various metal alloys and hard or flexible plastics. For example, the growth container 200 may comprise a metal structure, which in some embodiments can be constructed of stainless steel, supporting the mesh (e.g., 202, 204, 206). In some other embodiments, the growth container 200 may be constructed of hard plastic and the mesh 202, 204, 206 can be constructed of hard or flexible plastics. As demonstrated herein, such cylindrical growth container design can allow for maximal growth of fungal biomass with a minimal amount of inoculated substrate, in comparison to the conventional tray method.

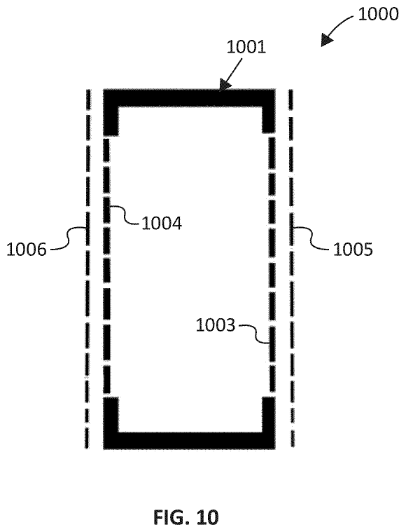

In various embodiments, the growth container 200 may further include a flexible and removable second mesh outermost to the first mesh (e.g., see 1005 and 1006 as shown in FIG. 10). However, such double mesh configuration is not limited to cylindrical growth containers and can be applied to any other shaped growth container (e.g., rectangular box). In such embodiments, the second mesh can be removed along with the fungal biomass by tearing or peeling the second mesh from the growth container (e.g., 200). The fungal product can then be removed or cut from the second mesh.

In various embodiments, the first mesh (e.g., attached to the container) and/or the second mesh (e.g., disposed on or spaced from the first mesh) can have a unitary or variable opening size. For example, the size of openings may be in the range of 0.5 mm to 25 mm. Various patterns of openings can also be employed, including random patterns. In some embodiments, the spacing for the openings of the first mesh and/or the second mesh can be in the range of 0.2 mm to 25 mm.

During cultivation (e.g., the incubation) fungal biomass grows around and through the mesh (e.g., 202, 204, 206) of the growth container 200 (e.g., see FIGS. 3A, 3C, 4A, 4B, 5, 6A, and 6B). As described herein, the mesh (e.g., 202, 204, 206) holds the inoculated substrate in place during cultivation and facilitates harvest of the fungal biomass.

The growth container 200 (e.g., illustrated in FIGS. 2A to 2D) can be configured to exchange nutrients and other material between the inoculated substrate (e.g., 215) and the growth chamber environment to facilitate maximum fungal growth. For example, the diameter of the growth container 200 may be small in comparison to its length. The bottom of the growth container 200 may be also made of a mesh, allowing growth of the fungal biomass at the bottom of the cylinder, and allowing water to drip from the substrate. The growth container 200 may be hung or otherwise placed vertically inside the growth chamber such that the bottom of the cylinder can be free for fungal growth. The growth container 200 may be maintained as a dynamic system, such as one that transitions between multiple positions (e.g., rotation along the longitudinal axis), increasing the exchange between inoculated substrate and the surfaces of the growth chamber. In various embodiments, several such growth containers 200 can be placed inside the growth chamber to maximize use of space.

As an example, FIG. 2C illustrates an example configuration where the growth container 200 with a tubing 250. The tubing 250 can be fed (e.g., at a center) through an interior 210 of the growth chamber or growth container(s), for example, under the substrate 215 (in FIG. 2B) so that liquid and/or gas flow 140 can be controlled to pass through. The tubing 250 can be a tube with holes 255 sized and spaced to release liquid or gas to the substrate 215. The holes 255 may be distributed along an entire circumference or portions of the circumference of the tubing 250. In an example, the tubing 250 can be used to irrigate the substrate 215 with water and/or provide a cooling mechanism. Fungal biomass being essentially made of water, adding water to the substrate 215 during the incubation phase allows for a higher yield of fungal biomass. Additionally, this tube can also be used to inoculate the substrate with a liquid inoculum before incubation, or to feed additional nutrients to the substrate, or gasses (such as CO₂) during incubation, as discussed above.

Further, as discussed herein, a tubing system 260 can be disposed within the cylindrical growth container 200 to deliver heat to the substrate (e.g., 215), as shown in FIG. 2D. In the illustrated embodiment, the tubing system 260 can be fed centrally through the growth container 200, for example, under the substrate 215 to deliver heat or coolant 145 in a controlled manner.

In some embodiments, as shown in FIG. 2E, the growth container 200 can be equipped with an auger screw mechanism 270 for filling and emptying the substrate (e.g., 215). The auger mechanism 270 can also allow mixing of sterile or inoculated substrate within the growth cylinder 200. In the illustrated embodiment, the auger 270 includes a central shaft surrounded by helical flutes 272. The auger 270 can be rotated in a first direction and/or second direction (e.g., clockwise and counterclockwise) to facilitate mixing, removal, or feeding of the substrate.

FIG. 2F illustrates a solid-liquid substrate system or SQUID 20. The SQUID system 20 can include a tubing system (e.g., 291) and a growth container 280 can be configured for supplying fluid (e.g., 284) to a substrate (e.g., 285). Such system 20 can involve a temporary immersion system involving a substrate (e.g., 285) periodically perfused with the fluid (e.g., 284). For example, the fluid (e.g., 284) may be liquid nutrient solution, liquid inoculum, water, or other liquid or semi-liquid for promoting growth of fungal biomass. This system 20 can facilitate increased growth speed due to submerged fermentation processes. Additionally, the ability to periodically expose a substrate to the fluid (e.g., liquid nutrient) allows the control of an amount and type of nutrients available to the fungal biomass. This in turn enables different types of substrates for fungal growth processes. For example, the substrate can be various side streams which in some embodiments may comprise water, lignocellulosic material, and supplements such as grain bran, hulls, husks, peels, leaves, stalks and/or flour. In some embodiments, the substrate may comprise a hydrolysate of agricultural byproducts. The substrate can be sterilized or pasteurized. The substrate can also be made of non-organic material, such as rockwool, vermiculite, silica beads, non-organic sponge, polymer foam or mesh, etc.

In the illustrated embodiment, referring to FIG. 2F, the SQUID system 20 can include a growth container 280 with a first compartment 281 at a bottom to hold fluid, a second compartment 282 configured to hold a substrate 285 above the first compartment, a tube 291 configured to deliver gas (e.g., pressurized air) to the first compartment 281. Additionally or alternatively, the growth container 280 may include a top cover 287 and a misting pipe 296 coupled to a misting system 295 for misting (e.g., water or liquid nutrients) the substrate 285 within the second compartment 282. In some embodiments, the container 280 can be a cylindrical container (e.g., 200) or a rectangular box (e.g., 100) including one or more meshes extending along circumference of the second compartment 282 so that the substrate 285 can be exposed to a growth environment. In some embodiments, the substrate 285 can be disposed in a sieve basket and placed over the first compartment 282.

The first compartment 281 and the second compartment 282 may be vertically separated from each other by a distance G1. In some embodiments, a raiser (e.g., a block sized to create the gap G1) with perforations can be used to separate the substrate 285 from the fluid 284. In some embodiments, the container 280 can be stepped (e.g., a stepped cylinder) so that the second compartment is larger than the first compartment 281 and shaped to include a step portion to support the substrate 285. In some embodiments, a perforated panel 286 may be provided between the first compartment 281 and the second compartment 282.

In some embodiments, the tube 296 can be a hollow tube configured to receive gas (e.g., pressurized air) at a top end portion and deliver the gas (e.g., pressurized air) from a bottom end portion immersed in the fluid 284 in the first compartment of the container. In some examples, the bottom end portion of the tube can include holes distributed around the circumference of the tube portion. In an example, gas can be pulsated or periodically passed through the tube. The gas supply can be controlled by an air controller 270. The gas enters the first compartment 281 causing the fluid 284 to displace upward towards the substrate 285 in the second compartment 282. The fluid 284 rises and comes in contact with a bottom portion of the substrate 285 and can be further absorbed by the substrate 285. Accordingly, the substrate 285 can receive water, liquid nutrients, liquid inoculum, etc. to facilitate optimal growth of fungal biomass from the substrate 285.

In some embodiments, the container 280 can include the cover 287 with a hole to receive the misting pipe 296. A first end portion of the misting pipe 296 can be connected to the misting system 295 and the second end portion can extend into the second compartment 282. The misting system 295 can include, but not limited to, an ultrasonic mister. The ultrasonic mister can be placed in a box and a rectangular hole cut into the box where a fan and filter are placed to push mist into the first end of the misting tube 296. In some embodiments, the second end portion of the misting tube 296 can include a valve to control an amount of misting to the second compartment 282 or the substrate 285.

As shown in FIGS. 3A-3B, one or more growth containers 300 can be arranged in an array within growth chamber 30. For example, a first set of containers 300 can be disposed within a first portion 310 of the growth chamber 30 and a second set of containers 300 can be disposed within a second portion 320 of the growth chamber 30. The present disclosure is not limited to a number of set containers and more than two set of containers can be included. The growth containers 300 can be examples of a cylindrical growth container 200, a growth box 100 (e.g., see FIG. 1A, 1B), or a combination thereof. In some embodiments, one end of the cylinder can be removable to allow for filling and emptying of the substrate (e.g., 215 in FIG. 2B). Each of the growth containers 300 can be vertically disposed within the growth chamber 30. For example, the growth containers 300 can be hung, via a cable 331 from a top side of the growth containers 300, as illustrated in FIGS. 3B and 3C. In other examples, the growth containers 300 can be suspended or supported using a rod, or other hanging means. The dimensions shown in the figures are only exemplary. In some embodiments, the number of growth containers can be 10, 20, 30, 50, 84, 90, or more to increase a growth surface for fungal biomass, and/or provide an energy efficient growth system.

FIG. 3B illustrates a side view of an array of containers 300 disposed within the first chamber portion 310 of FIG. 3A. The containers 300 can be arranged in rows and columns. For example, the containers 300 can be arranged in chamber sub-portions 302, 304, and 306. These chamber sub-portions 302, 304, 306 can be vertically spaced from each other. In the illustrated example, the first chamber portion 310 can also include a cooling and misting system 350 configured to maintain an environment conducive to fungal biomass growth.

Referring to FIG. 3C, each growth container 300 (used in FIGS. 3A and 3B) can be a cylindrical growth container having a length L1 (e.g., 1.5 m to 2 m long, preferably 1.5 m) and a diameter D1 (e.g., 0.25 m to 0.35 m, preferably 0.3 m). The length L1 can be in a range 1.5 m to 2 m, preferably 1.5 m or other lengths. The diameter D1 can be in a range 0.25 m to 0.35 m, preferably 0.3 m or other diameters. In some examples, a ratio of length L1 to diameter D1 can be selected to provide maximal surface area for fungal biomass growth, maximize a number of cylinders within the growth chamber 30, or may be based on other fungal biomass growth factors.

The growth container 300 can be configured to facilitate fungal biomass growth on two longitudinal sides 312, 314. For example, meshes may be provided on the longitudinal sides 312, 314 so that fungal biomass 313, 315 can grow from the substrate in the container 300 and through the meshes at the sides 312, 314. In the illustrated embodiments, fungal biomass 313, 315 can grow in a generally horizontal direction. The length of the fungal biomass growth 313, 315 can be limited to D2, D4 (e.g., 0.04 m, 0.045 m, 0.05 m or other predetermined length). Accordingly, the distance between the growth containers 300 (e.g., in chamber portion 302 illustrated in FIG. 3B) can be more than twice of D2 or D4. For example, a distance between the growth containers 300 can be such that a gap of 0.05 m exists between adjacent fungal biomasses so that an appropriate environment can be provided between growth containers 300, fungal biomass can be easily harvested, or other growth or harvesting related factors.

Although examples herein illustrate vertical orientation and other orientations are possible without limiting the scope of the present disclosure. As another example, the growth container (e.g., 200) may be hung horizontally (not illustrated) from both of its two ends, each end being covered with mesh. For example, a horizontal orientation refers to a container oriented such that its longitudinal axis (e.g., length L1) is oriented along a horizontal direction.

FIGS. 4A-4B illustrates a growth system employing a rotation system 410 allowing rotation or linear movement (e.g., tilting) of the growth container(s)) during incubation. For example, FIG. 4A illustrates a growth system 40 including a cylindrical growth container 200 coupled to the rotation system 410. FIG. 4B illustrates a growth system 50 including a rectangular box 430 coupled to the rotation system 410. The rotation system 410 may include a motor 401 coupled to a gearbox 403 to rotate a shaft 405 in a specified direction (e.g., R1) at a specified speed (e.g., w). Accordingly, the growth container 200 or 430 can be rotated to expose its surface of growth environment for maximum fungal biomass growth 420. In some embodiments, the rotation system 410 can be employed for harvesting the fungal biomass 420, as discussed later in the disclosure.

Fungal growth is guided by gravitropism. During fruiting body formation, hyphae first grow negative gravitropic (stipe) and later positive gravitropic (hymenium). Based on the gravitropism effect, a rotational or translational movement of the growth container inside the growth chamber could trigger fungal growth and possibly avoid fruiting body formation, thereby increasing mycelium production. Dynamic systems can improve mycelial yield in relation to decreasing fruiting body formation by incorporating controlled rotational/translational movement of the growth container(s) to avoid triggering fruiting body formation. In still other embodiments, growth containers can be manually rotated or tilted during incubation.

In an aspect of this disclosure, a method for growing fungal biomass in a growth chamber (e.g., 15 in FIG. 1) is provided. The method may comprise providing the fungal biomass growth system as disclosed herein and incubating the growth container(s) in the growth chamber under conditions suitable for growing fungal biomass.

In various embodiments, the method may comprise preparing a spawn as a grain mixture, a sawdust mixture, or a solution containing an isolated and specific fungal species. In various embodiments, the genus of fungal is selected from: Aspergillus (e.g., A. oryzae), Flammulina (e.g., F. velutipes), Fusarium (e.g., F. venenatum), Ganoderma (e.g., G. lucidum, G. sessile), Grifola (e.g., G. frondosa), Hericium (e.g., H. erinaceus), Kuehneromyces (e.g., K. mutabilis), Laetiporus (e.g., L. sulphureus), Lentinula (e.g., L. edodes), Morchella (e.g., M. esculenta), Neurospora (e.g., N. crassa), Pholita (e.g., P. adiposa, P. nameko), Pleurotus (e.g., P. djamor, P. eryngii, P. ostreatus, P. pulmonarius), and Rhizopus (e.g., R. oligosporus). The spawn is mixed with a substrate, which in some embodiments may comprise water, lignocellulosic material, and supplements such as grain bran, hulls, husks, peels, leaves, stalks and/or flour. In some embodiments, the substrate may comprise a hydrolysate of agricultural byproducts. The substrate is sterilized or pasteurized before inoculation with the spawn. In various embodiments, the ratio of spawn to substrate is from about 1.5% to about 10% by weight.

The mix of spawn and substrate is left to pre-colonize and incubate in the dark at a temperature in the range of about 20° C. to about 28° C. for 2 days (e.g., about 48 hours) to about 7 days before it is broken down and packed in sterilized growth containers (as described). When placed in the growth containers, the substrate can be in sufficiently solid state to be held in place by the mesh. The filled growth container can be placed vertically or hung in the growth chamber, in such a way that the air flow can be brushing each side of the container. That is, the substrate mass can be surrounded by air. In various embodiments, the growth container(s) are incubated in the growth chamber for at least about 2 days, or at least about 3 days, or at least about 4 days, or at least about 5 days, or at least about 8 days, or at least about 10 days, or at least about 12 days. For example, the growth container(s) are incubated in the growth chamber for about 5 days to about 14 days, or from about 5 days to about 10 days. During the incubation period, water can be fed to the inside of the growth container(s), for example, using the irrigation system described herein. The growth chamber provides a closed space where gas level (e.g., CO₂ level, O₂ level, N₂ level or other gases), temperature, air flow, relative humidity and light are controlled. In various embodiments, O₂ levels can be controlled within a specified range for optimal growth of the fungal biomass. In various embodiments, the incubation parameters maintained inside the growth chamber range from about 1% to about 10% CO₂, from about 15° C. to about 35° C., and a relative humidity from about 80% to about 100% (or other range as described herein). In various embodiments, air flow can be activated and piloted inside the growth chamber to brush all exposed surfaces of the substrate. The inside of the chamber can be completely dark, but lights of different wavelengths can be turned on periodically.

Accordingly, the growth chamber with the growth containers herein can be configured to have a substantial impact on the growth yield of the fungal biomass, while allowing for an efficient harvesting process that produces quality fungal biomass suitable for use as or in food products.

The present disclosure further describes a harvesting system and methods of harvesting fungal biomass e.g., mycelia biomass from a growth container having surfaces exposed to a growth chamber environment. The harvesting process, in various embodiments, includes shearing, reaping, threshing, gathering, and winnowing using a harvesting system. The harvesting system can include a variety of agricultural implements suitable for cultivation such as shears, cutters, etc. In various embodiments, harvesting can occur vertically (see, e.g., FIGS. 2A, 2C-E, 5, and 6A-6B) using gravitation to remove the fungal mass from a substrate surface. Further, the harvesting system can include post-harvest collection. The fungal biomass can be harvested at a specified growth stage such as mycelium forming stage, primordia forming stage, fruiting body forming stage, or any intermediate stage. The present disclosure is not limited to a particular fungal biomass, or time of harvesting. The harvesting systems herein can be engaged at specified growth stages or an instance in time. The harvesting systems herein can be used for a particular type of fungal biomass or a hybrid of the fungal biomass depending on whether the substrate inoculated for growth of one or more type of fungal biomasses.

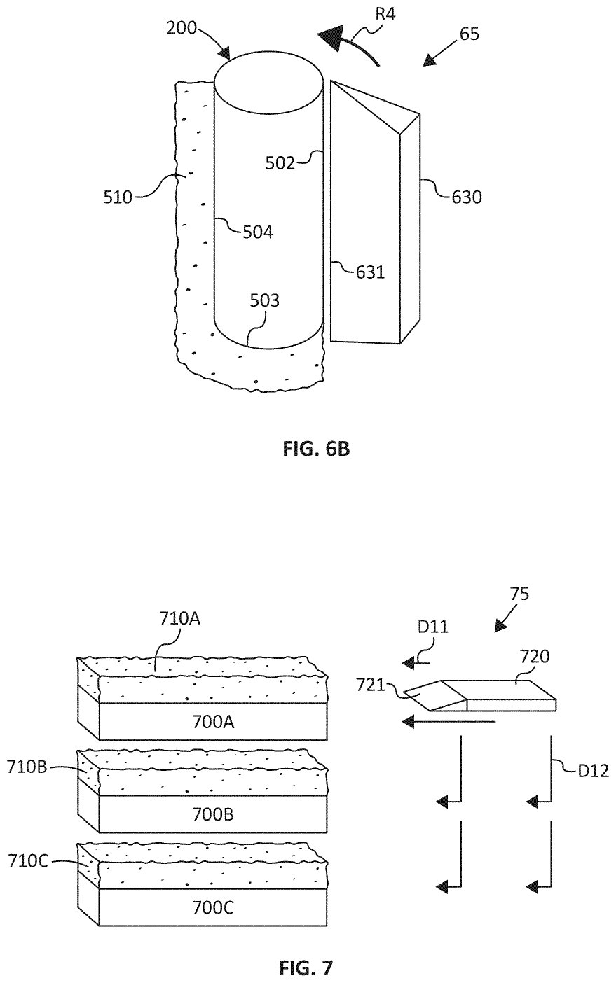

In various embodiments, the harvesting systems and processes can be configured such that a harvesting mechanism (e.g., a cutter) can be stationary and a growth surface of a growth container can be positioned such that a fungal biomass mat can be sheared from the growth surface (e.g., including a mesh) as they are positioned about the harvesting mechanism (see, e.g., FIG. 6A). In various embodiments, the mycelial mass can be kept stationary and the harvesting mechanism moves about the growth surface to shear the mycelial mat from the growth surface (e.g., including a mesh), as shown in FIGS. 5, 6B, and 7. Alternatively, both the harvesting mechanism and growth surface are configured to move relative to one another.

FIG. 5 illustrates a harvesting system 50 for harvesting fungal biomass (e.g., mycelium) 510 from a cylindrical growth container 200. In the illustrated embodiment, the harvesting system 50 includes a movable cutter 520 configured to move relative to the growth container 200 (which can be cylindrical, rectangular, hexagonal, or higher order polynomial shapes). For example, the movable cutter 520 can be configured to move in a direction D10 e.g., vertically or parallel to a longitudinal surface (e.g., 502 or 504) of the container 200 with fungal biomass 510. The movable cutter 520 can include a cutting edge 521 with a tip oriented to shear the fungal biomass 510 as it moves along the surface 502 or 504 of the cylindrical growth container 200. Additionally or alternatively, the cylindrical growth container 200 can include a bottom mesh 503 through which the fungal biomass 510 can grow. The movable cutter 520 can be oriented to shear and remove the fungal biomass from the bottom mesh 503. In some embodiments, one or more movable cutters 520 can be disposed between the growth containers 300 (e.g., within the chamber portions 302, 304, 306 in FIG. 3B) for harvesting the fungal biomass grown thereon.

FIG. 6B illustrates another example of a harvesting system 65 including a movable cutter 630 for harvesting the fungal biomass 510 from a cylindrical growth container 200. In the illustrated embodiment, the movable cutter 630 can be configured to rotate along a direction R4 (e.g., clockwise or counter clockwise). Additionally or alternatively, the cutter 630 can be moved radially towards or away from the growth surface 502 of the container 200. The movable cutter 630 can include a cutting edge 631 that can be oriented and aligned along the growth surface (e.g., 502) of the container 200 to shear the fungal biomass 510. In some embodiments, the movable cutter 630 can include another edge configured to cut the fungal biomass 510 from a bottom surface 503 of the growth container 200.

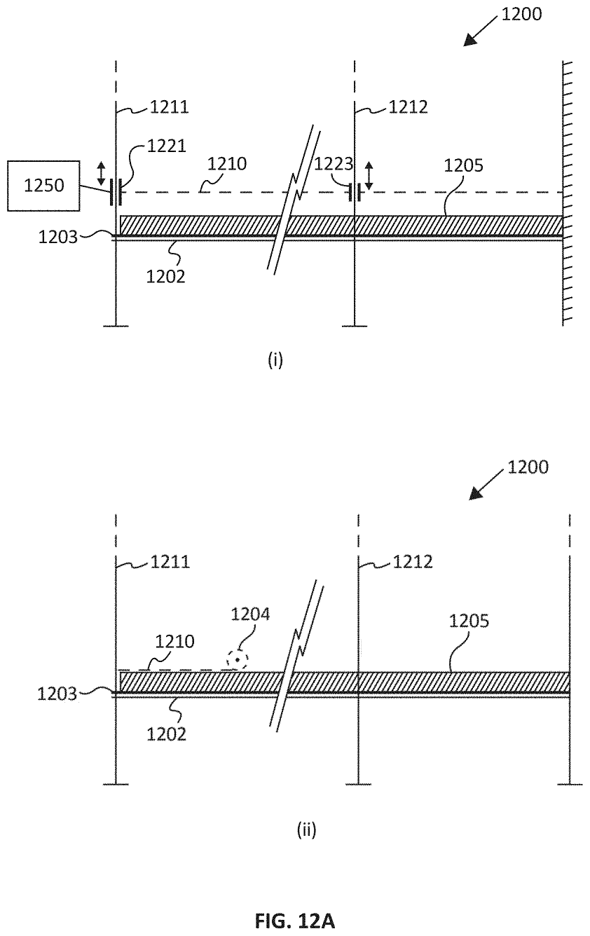

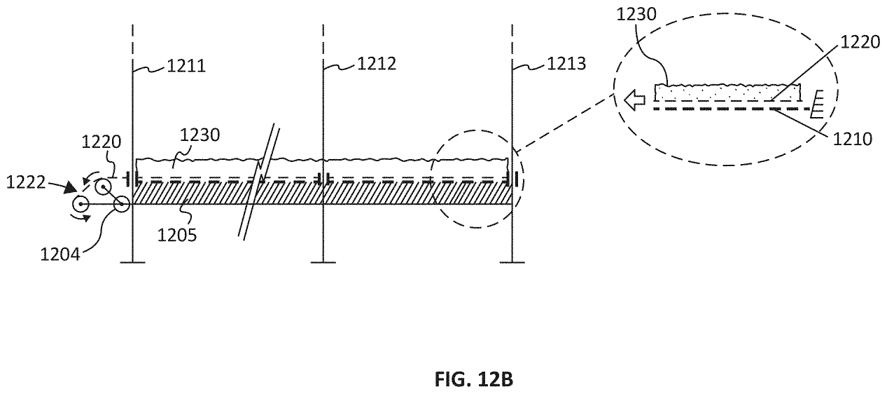

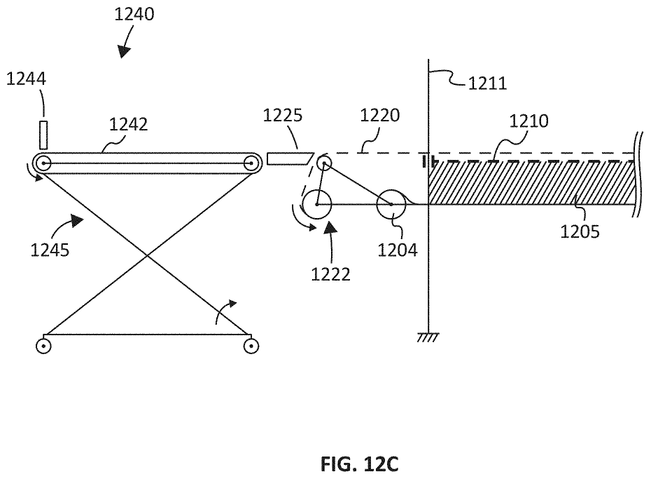

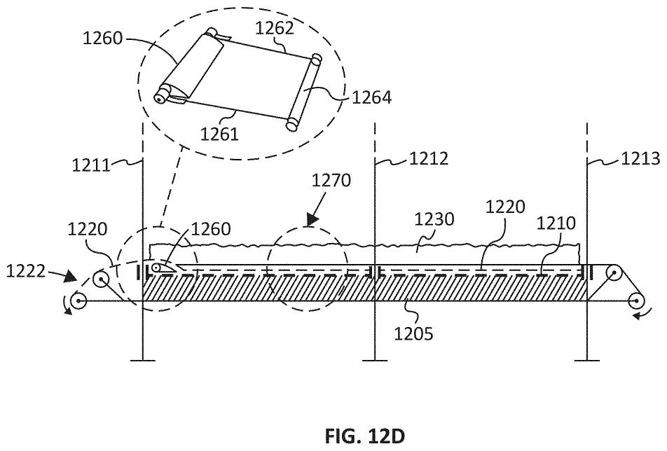

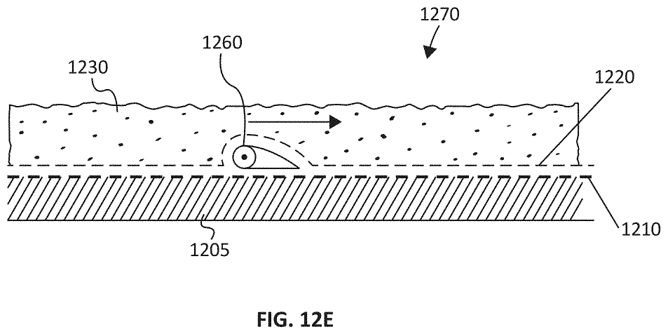

FIG. 7 illustrates a harvesting system 70 for harvesting the fungal biomass from one or more horizontally oriented growth containers (e.g., 700A, 700B, 700C) or planar system (see FIGS. 12A-12E). In the illustrated embodiment, the harvesting system 70 includes a movable cutter 720 configured to move (e.g., slide) relative to the growth containers 700A, 700B, and 700C. For example, the movable cutter 720 can be configured to move in a first direction D11 e.g., horizontally or parallel to a longitudinal surface of a first container 700A to harvest the fungal biomass 710A thereon. The movable cutter 720 has an edge 721 with a tip. As an example, the edge 721 can be a sharp edge, two sharp parallel blades (e.g., like electric knife) with teeth to cut through the fungal biomass, an edge like a shearing knife, or other cutting edges. The movable cutter 720 can be oriented such that the tip portion of the edge 721 can be engaged with the fungal biomass 710A and shear the fungal biomass as the cutter 720 moves along the first direction D11. Additionally or alternatively, the movable cutter 720 can be configured to move along a second direction D12 (e.g., vertically) and align with a second container 700B or a third container 700C. Once aligned, the cutter 720 can be moved in along the first direction D11, as discussed above, to harvest fungal biomass 710B or 710C from the respective containers 700B or 700C.

FIG. 6A illustrates a harvesting system 60 including a cutter 620 and a movable (e.g., rotatable) growth container 200. As illustrated, the growth container 200 can be coupled to a rotation system (e.g., similar to the system 410 in FIG. 4A-4B) including a motor 601 and a shaft 603. The cutter 620 may include an elongate cutting edge 621. The cutter 620 can be moved radially to contact the cutting edge 621 with a growth surface 502 on which the fungal biomass 510 is grown. Once positioned, the cutter 620 may be held stationary and the motor 601 may rotate the shaft 603 to rotate the growth container 200 in a direction R3 (e.g., clockwise or counter clockwise). As the growth container 200 rotates, the cutting edge 621 causes the fungal biomass 510 to shear off the growth surface 502, 504.

The present disclosure provides fungal biomass harvesting systems enabled by mesh systems described herein. In various embodiments, these systems and methods allow for a more efficient and automatable process of harvesting the mycelial biomass from the substrate, as well as an improved quality of the harvested biomass, in comparison to conventional processes.

In some embodiments, the mycelial biomass can be grown through and around a mesh for a growth container (e.g., 200). The mesh separates the inoculated substrate from the chamber environment. In these embodiments, the mycelial biomass can be cut from the mesh for harvest. For example, a metal cutter can be slid along the surface of the mesh (e.g., slid along the external surface of the growth container having the mesh). In some embodiments, for harvest, growth containers or separate sections of the growth containers (e.g., mesh panels of the box) can be pulled through a fixed cutter to thereby separate the biomass from the mesh and substrate. In other embodiments, the growth container can be substantially cylindrical or conical, and the growth container can be rotated along its axis with the cutter placed against the mesh to separate the fungal biomass from the growth container.

FIG. 8A illustrates an example of a mesh-based harvesting system 800. The harvesting system 800 can include a flexible removable mesh 803 disposed over a growth surface of a growth container 802. The growth container 802 can be rectangular (e.g., 100, 802) or cylindrical (e.g., 200, 300) in shape and configured to hold a substrate 805. Fungal biomass 804 can grow from the substrate through the mesh 803. The flexible mesh 803 allows for simple peeling of the grown mycelial biomass 804 from the growth container 802. In some embodiments, the mesh 803 can be fully detached from the substrate, that is, portions of the substrate 805 do not remain with the grown mycelial biomass 804 after peeling the flexible mesh 803 from the growth container 802. The mesh 803 can be cleaned and reused. Alternatively, the mesh 803 can be discarded.

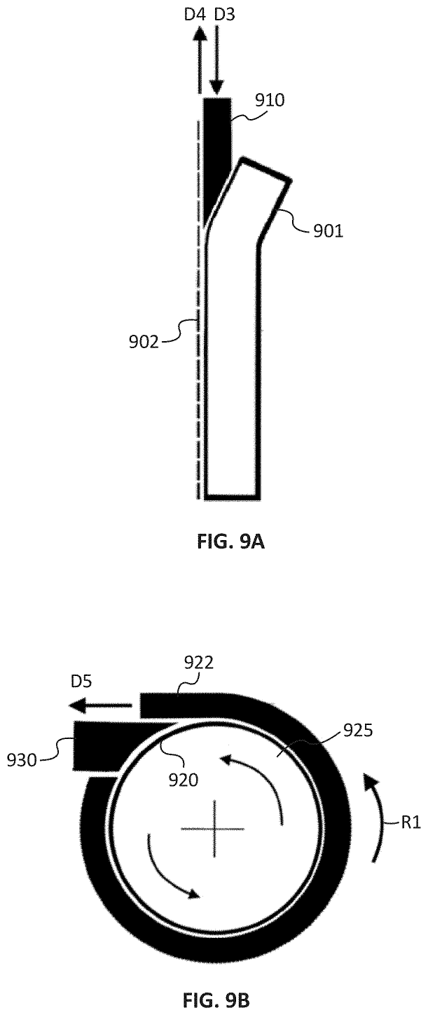

In some embodiments, referring to FIGS. 8B, 9A, and 9B, a mesh and mycelial biomass that grew on top of the mesh can be separated in a semi-automated or fully automated process. For example, as shown in FIG. 8B, such a harvesting system can include a slidable mesh 811 and a cylindrical roller 812. The mesh 811 can be pulled and wrapped around the cylindrical roller 812 due to its flexibility. Furthermore, the system may include a separating cutter 815 configured to remove the mycelial biomass 804 from the slidable mesh 811. This can provide a quality biomass harvest with no substrate attached to the mycelial biomass (e.g., 805). Accordingly, the harvested biomass 804 can be safe for use as or in food products intended for human consumption. The mesh 811 can then be cleaned and reused. Alternatively, the mesh 811 can be discarded.

FIGS. 9A-9B illustrates harvesting mycelial biomass from mesh using a cutter, according to some embodiments. For example, in FIG. 9A, a substrate 901 can grow through a mesh 902. A cutter 910 can be configured to slide along the mesh 902 to separate the mycelial biomass 901. The cutter 910 can be advanced in a first direction D3 against a length of the substrate 901 and/or retracted in a direction D4 opposite to the direction D3. In a vertically oriented container and vertically oriented mesh 902, the cutter 910 can move vertically downward D3 causing the separated mycelial biomass 901 to advantageously fall under gravity and be collected in e.g., a tray or pan placed underneath the container. Also, advantageously, the collection pan or tray can be placed on a conveyor so that the harvested mycelial biomass 901 can be easily transported to a specified delivery area. Thus, no additional mycelial biomass handling equipment may be needed. The separated biomass 901 does not have any portion of substrate on the harvested mycelial biomass 901. In another example, as shown in FIG. 9B, a growth system can include a cylinder 925 along which a mesh 920 may be rotated. The mycelial biomass 922 can grow around an entire circumference or circumferential portions of the cylinder 925 and through the mesh 920. The cylinder 925 can be a roller to roll the mesh 920 or a cylindrical growth container (e.g., 200). A cutter 930 can be oriented at a circumference of the mesh 920 such that a tip of the cutter 930 cuts the mycelial biomass 922 as the cylinder 925 rotates. Once aligned, the cutter 930 can stay fixed with respect to the cylinder 925. The harvested mycelial biomass 922 can be pulled over the cutter 930 along a direction D5 (e.g., horizontal direction) and further collected.

FIG. 10 illustrates a double mesh system 1000 for growing and harvesting mycelial biomass from a growth container 1001, according to some embodiments. The growth container 1001 may comprise two meshes: one or more first removable meshes 1005, 1006 on an outermost side that can be flexible and designed to facilitate the harvest, and one or more second meshes 1003, 1004 that can be built into the growth container 1001, according to various embodiments. The built-in meshes 1003, 1004 stays attached to the growth container 1001 to hold a substrate (not illustrated in FIG. 10) in place during harvesting, while the mycelial biomass (not illustrated in FIG. 10) sticks to the removable mesh 1005, 1006 for processing. This system 1000 can increase the process efficiency and positively affects the quality of the product as no part of the substrate will be present on the fungal biomass.

It can be understood that the present disclosure is not limited to particular cutter, mesh system, or growth containers, as discussed above. Depending on the design of the growth container, the cutter shape and form can change. For example, the cutter can include an inclined straight edge (e.g., see FIG. 9A), a curved edge. (e.g., see FIG. 9B), blades with teeth, or other types of cutter. As discussed, a growth cylinder or cone can be employed, where the cylinder or cone can be rotated along its axis with the cutter placed against the mesh, to separate or peel the fungal biomass from the growth cylinder. The rotating motion of the growth cylinder or cone can advantageously allow for a cautious separation of the fungal biomass from the mesh, and the technique can easily be automated, allowing for an efficient and safe process at scale.

FIG. 12A illustrates a sectional view of a substantially planar bed-based growth and harvesting system 1200 in accordance with some embodiments. The system 1200 can include one or more sets of vertically disposed beds (e.g., 1202) or shelves which can hold or support a substrate configured for growing fungal biomass. In some embodiments, growth containers holding the substrate therein can be placed on the shelves. A substrate 1205 can be loaded on the bed 1202 using a substrate loader a first end (e.g., at a right side) and a harvesting system can be disposed at a second end (e.g., at a left end) opposite to the first end. The harvesting system is further discussed with respect to FIGS. 12B and 12C. The substrate loader can be a conveyor e.g., including a conveyor belt rotatable via rollers.

The system 1200 can include the substrate 1205, and a first mesh 1210 that can be positioned over the substrate 1205. The substrate 1205 can be placed on or within a flexible net a perforated membrane, or a cloth bag that can be pulled onto a shelf. In another example, the substrate 1205 can be disposed in a growth container (e.g., a tray or flexible cover) where a top surface is exposed to a growth chamber environment to facilitate fungal biomass growth. In some embodiments, the substrate 1205 can be positioned over an elongated bed 1202 using the substrate loader. The elongated bed 1202 can be made of rigid material such as metal (e.g., aluminum, steel, etc.) or wood. The substrate loader can be inclined relative to the bed 1202 so that the substrate 1205 can easily slide assisted by gravity and motion of the conveyor belt.

In an example, a substrate loading system can include a net 1203 or cable couplable to a net pulling means such as a roller. One end portion of the cable 1203 can be attached to an end of a net or a container holding the substrate 1205 and another end portion can be attached to the net pulling means. As the roller rotates, the substrate 1205 can be pulled over the bed 1202.

In some embodiments, as illustrated in FIG. 12A(i), the first mesh 1210 can be configured to move vertically over the substrate 1205. For example, the first mesh 1210 can be movable coupled to vertical columns e.g., 1211, 1212, 1213 (in FIG. 12B). Each column can include a slider to effectuate the vertical movement of the first mesh 1210. For example, sliders 1221, 1223 can be slidably coupled to the columns 1211, 1212, respectively, and further configured to support the first mesh 1210. The vertical columns 1211, 1212 can be spaced from each other and oriented perpendicular to the bed 1202. The first mesh 1210 can be made of aluminum or a flexible material. In some embodiments, the first mesh 1210 (in FIG. 12A(ii)) can be a flexible mesh (nylon, aluminum, plastic, stainless steel, wire mesh or perforated sheet) that can be rolled over the substrate and tighten on each end of the bed to press the substrate down. For example, the first mesh 1210 can be rolled over the substrate 1205, manually or with an electrical rolling system 1204. Further, the first mesh 1210 can be coupled to cables or cords that can be coupled to one or more vertical columns 1221, 1212 to tightly spread the first mesh 1210 over the substrate 1205. The first mesh 1210 can include holes sized to allow fungal biomass to grow therethrough.

FIG. 12B and FIG. 12C illustrate a mesh configured for harvesting fungal biomass from the planar bed system 1200. The harvesting subsystem of the system 1200 can include a second mesh 1220 and a mesh roller 1222 coupled to the second mesh 1220. The second mesh 1220 can be disposed over the first mesh 1210 and extend along a length of the substrate 1205. In some embodiments, the second mesh 1220 can be rolled on top of the first mesh 1210 and tightened (e.g., using cords, springs, or other tightening means) over the first mesh 1210. Fungal biomass 1230 can grow from the substrate 1205 through both the meshes 1210, 1220.

The mesh roller 1222 coupled to the second mesh 1220 and configured to move the second mesh 1220 relative to the first mesh 1210 causing the fungal biomass 1230 to shear. For example, the relative motion between the meshes 1210 and 1220 creates a scissoring effect which shears the fungal biomass 1230, which further sits on the second mesh 1220. The mesh roller 1222 can be configured to translate the second mesh 1220 around and toward the bottom side of the bed 1202. As mentioned earlier, on the bed 1202, a growth container with the substrate 1205 can be placed.

The present disclosure is not limited to the mesh roller 1222 for moving the second mesh 1220. In another example, a slider 1250 (e.g., see FIG. 12A) may be configured to pull the second mesh 1220 approximately parallel to the substrate 1205 so that the fungal biomass 1230 can be separated from the substrate 1205 onto the second mesh 1220. The slider 1250 may be operated manually or via a pulling mechanism, such as the mesh rollers 1222 may be omitted in such embodiment.

In some embodiments, referring to FIG. 12C, the harvested fungal biomass 1230 on the second mesh 1220 can be further transported to a delivery area 1240. The delivery area 1240 can include a platform 1242 proximate to the second end (e.g., the left side) of the system 1200. The platform 1242 can further include a stopper 1244 located on the platform at an end away from the substrate 1205. The stopper 1244 can be a rigid panel extending perpendicular to the platform 1242 to stop the fungal biomass 1230 from advancing off the platform 1242 while forming a fungal biomass layer on the platform for easy handling. In some embodiments, a cutter can be included between the platform 1242 and the bed 1202 to cut the fungal biomass. This allows cutting several sections of the fungal biomass that can then be handled and removed from the platform 1242, so that more can be loaded from the second mesh 1220.

In some embodiments, the harvesting system can include a cutter 1225 disposed between the second end of the substrate 1205 and the platform 1242 to separate the fungal biomass 1230 on the second mesh 1220 while the second mesh 1220 wraps around the mesh roller 1222 and moves under the substrate 1205. The cutter 1225 can be an elongated cutter including a cutting edge oriented against the second mesh 1220 and a top surface over which the fungal biomass 1230 can be moved from the second mesh 1220 to the platform 1242.

In some embodiments, the platform 1242 can be further coupled to a lift system 1245 or a conveyor to convey the harvested fungal biomass layer to a specified location. The lift system 1245 can be configured to move the platform up or down to a specified height. The lift system 1245 can be a scissor mechanism or a hydraulically actuated shaft configured to move the platform 1242 in up or down direction. Further, an operator, robot, or conveyor can remove the harvested fungal biomass layer on the platform 1242.

FIG. 12D illustrates another example of harvesting that can be employed in the planar bed system 1200. FIG. 12E illustrates an enlarged portion with a cutter of the planar system 1200 of FIG. 12D. As illustrated, a cutter 1260 can be disposed between the first mesh 1210 and the second mesh 1220 of the planar system 1200. The cutter 1260 can include an elongated edge extending along a width of the bed 1202. The cutter 1260 can be configured to move relative to the first mesh 1210. For example, the cutter 1260 can slide between the meshes 1210 and 1220. As illustrated, the cutter 1260 can move along a length of the bed 1202 from left to right. In some embodiments, the cutter 1260 can slide in a direction opposite to the movement of the second mesh 1220. As illustrated in FIG. 12A, the cutter 1260 can be coupled to pulling cables 1261 and 1262. The cables 1261, 1262 can be further coupled to a cable roller 1264 configured to rotate and pull the cables 1261, 1262. For example, the cable roller 1264 can be driven by a motor, or manually by a crank handle. As the roller 1264 rotates e.g., in clockwise direction, the cables 1261, 1262 pull the cutter 1260 towards the right causing the fungal biomass 1230. The cutter 1260 traversing between the two meshes 1210, 1220 allows to reduce pulling forces applied to the second mesh 1220 to shear the fungal biomass 1230. Also, the cutter 1260 can facilitate use of a flexible or thinner mesh (e.g., 1220) than used in FIG. 12B.

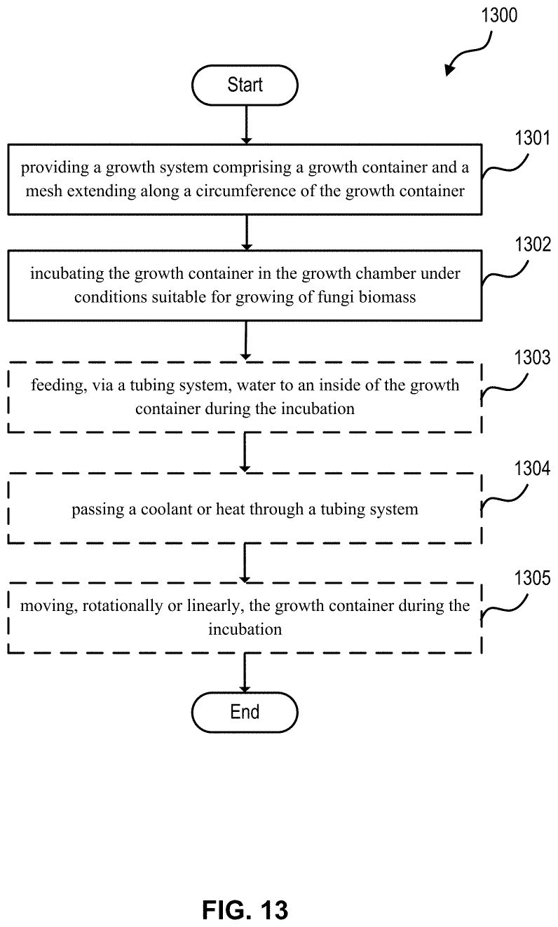

FIG. 13 is a flow chart of a method 1300 for growing a fungal biomass (e.g., mycelium) using containers and growth environment discussed herein. As an example, the method 1300 can include steps 1301 and 1302. Step 1301 can involve providing a growth system. For example, the growth system can include a growth container (e.g., 100, 200, 300) holding an inoculated substrate (e.g., 110, 215), and a mesh extending along a circumference of the growth container and configured to expose the inoculated substrate to a growth chamber environment and allow growth of fungal biomass through the mesh. The meshes (e.g., see FIG. 1B, 2A) can extend vertically. The growth container can be a vertically oriented cylinder (e.g., 200) or a rectangular box (e.g., 100) allowing growth of the fungal biomass along a vertically oriented longitudinal side of the cylinder or the rectangular box. The longitudinally extending meshes (e.g., see FIG. 1B, 2A) can expose a large surface area of the substrate to the growth environment to facilitate optimal use of the growth container for growing the fungal biomass. In some embodiments, one or more growth containers (e.g., 100, 200) may be disposed within the growth chamber as shown in FIGS. 3A-3B. The growth containers may be arranged in an array to maximize use of the space within the growth chamber.

Step 1302 can involve incubating the growth container (e.g., 100, 200) in the growth chamber under conditions suitable for growing of fungal biomass. For example, as discussed herein, the growth chamber can be configured to provide one or more controlled growth parameters. For example, during incubation, the growth chamber can be supplied with controlled levels of gasses, including but not limited to, carbon-dioxide, oxygen, nitrogen, and/or other gasses. The growth chamber may be further provided with controlled temperature, controlled air flow, and/or controlled relative humidity. As an example, the method may involve controlling the growth chamber environment so that the growth chamber can include from about 0.04% to about 10% CO₂, a temperature of from about 15° C. to about 35° C., a relative humidity from about 80% to about 100%, or a combination thereof. In some embodiments, during incubating, air flow can be activated and piloted inside the growth chambers to brush surfaces of the growth containers that expose the inoculated substrate, e.g., see FIGS. 1, 1A. In some embodiments, the growth chamber can be a hermetic chamber and enable total darkness. In some embodiments, the growth chamber provides a controllable or programmable lighting system of different wavelengths. In some embodiments, which include an array of growth containers (e.g., 3A) can advantageously provide an energy efficient solution. For example, the amount of CO₂ released by the substrate during incubation can naturally facilitate growth of fungal biomass with minimal or no additional CO₂ pumping into the growth chamber.

In some embodiments, the method 1300 can further include a step 1303 for feeding, via a tubing system, water to an inside of the growth container during the incubation. For example, FIGS. 1C and 2C illustrate examples of the tubing systems for rectangular box and cylinder, respectively. In some embodiments, the method 1300 can further include a step 1304 for passing a coolant or heat through a tubing system inside the growth container during the incubation. For example, FIGS. 1D and 2D illustrate examples of the tubing systems for rectangular box and cylinder, respectively. In some embodiments, the method 1300 can further include a step 1305 for moving, rotationally or linearly, the growth container during the incubation. For example, FIGS. 4A and 4B illustrate examples of the rotation systems for rectangular box and cylindrical containers.

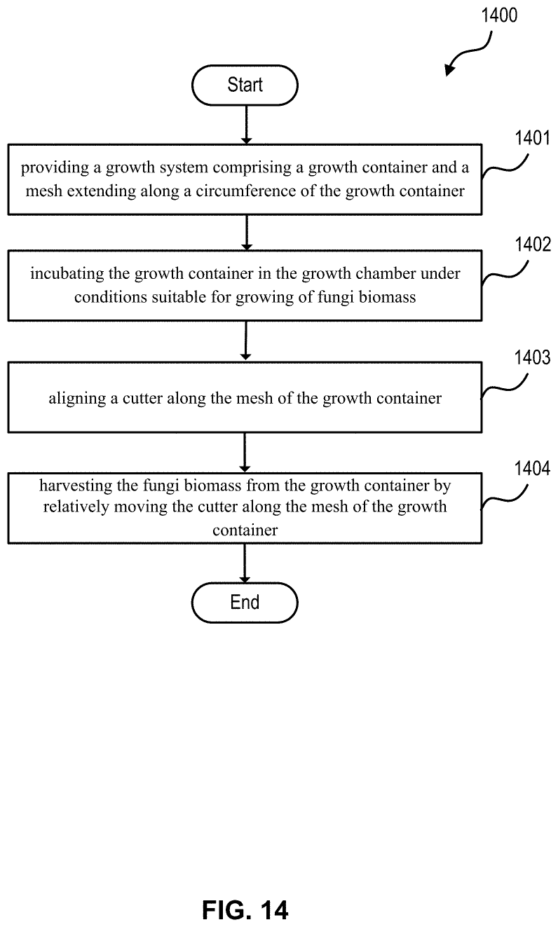

FIG. 14 is a flow chart of a method 1400 for harvesting fungal biomass. The harvesting method 1400 can advantageously provide harvested fungal biomass that contains minimal to no portions of the inoculated substrate. As an example, the method 1400 can involve steps 1401, 1402, 1403, and 1404. Step 1401 can involve providing a growth system including a growth container holding an inoculated substrate and a mesh extending along a circumference of the growth container and configured to expose the inoculated substrate to a growth chamber environment and allow growth of fungal biomass through the mesh. Examples of growth containers can be 100, 200, 300, as discussed herein e.g. see FIGS. 1-4. In various embodiments, the growth container can be a vertically oriented cylinder or a vertically oriented rectangular box allowing growth of fungal biomass along a longitudinal side of the cylinder or the rectangular box. For example, as shown in FIG. 1B, the rectangular box 100 may be vertically oriented such that the meshes 102 and 104 extend in vertical planes.

Step 1402 can involve incubating the growth container in the growth chamber under conditions suitable for growing the fungal biomass, as discussed herein. After the fungal biomass grows around the growth container, the fungal biomass can be harvested. For example, fungal biomass can be harvested at different growth stages or points in time as desired.

Step 1403 can involve aligning a cutter along the mesh of the growth container. Examples of cutters are illustrated in FIGS. 5, 6, 7, 8B, 9A and 9B. The cutter can include a cutting edge and a tip. Aligning the can involve aligning the tip such that the cutting edge when moved relative to the mesh causes the fungal biomass to shear and be separated.

Step 1404 can involve harvesting the fungal biomass from the growth container by relatively moving the cutter along the mesh of the growth container. In some embodiments, the harvesting can involve sliding the cutter along a longitudinal side of the growth container. For example, as shown in FIG. 5, the cutter 520 can be moved downward to cut mycelium from the cylindrical growth container. FIGS. 7 and 9A show other examples of sliding the cutter relative to the mesh on which fungal biomass growth exists.

In some embodiments, the harvesting can involve pulling sections of the growth container through a fixed cutter separating the fungal biomass from the mesh and the substrate. In some embodiments, the harvesting involves rotating the growth container along its central axis with the cutter placed against the mesh to separate the fungal biomass from the growth container. For example, as shown in FIG. 6A, the cylindrical container 200 is rotated about a central axis 603 and the cutter 620 harvests fungal biomass from the circumference of the cylinder 200. In some embodiments, the growth system can include a second mesh surrounding the mesh at the circumference of the growth container. In such embodiments, harvesting can involve moving the second mesh from the growth container to cut the fungal biomass from the mesh; and removing the fungal biomass from the second mesh. For example, FIG. 10 shows a growth container with a built-in mesh and a second mesh disposed over the built-in mesh such that the second mesh can slide or peeled off to remove any fungal biomass grown thereon.

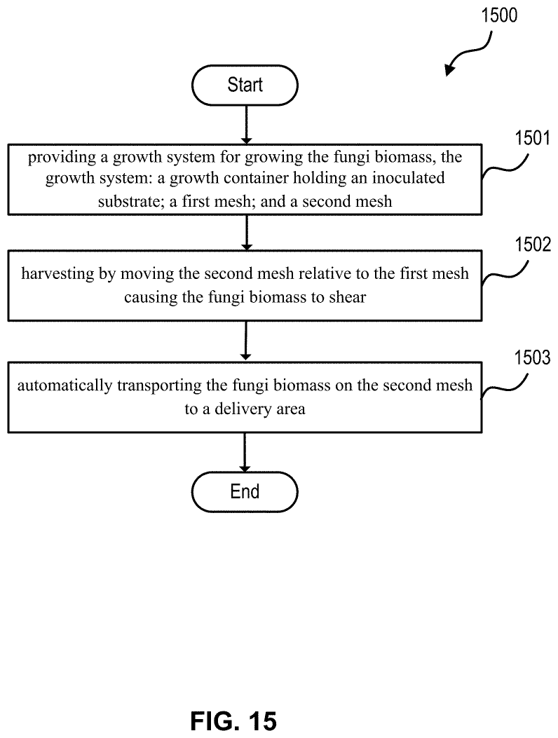

FIG. 15 is a flow chart of a method 1500 for harvesting fungal biomass. The harvesting methods herein advantageously provide harvested fungal biomass that contains minimal to no portions of the inoculated substrate. As an example, the method 1500 can involve steps 1501, 1502, and 1503.

Step 1501 can involve providing a growth system for growing the fungal biomass. The growth system can include a growth container holding an inoculated substrate; a first mesh coupled to the growth container and extending over the inoculated substrate such that the fungal biomass grows from through the first mesh; and a second mesh disposed over the first mesh such that the fungal biomass grows further through the second mesh. For example, as shown in FIG. 12A, the growth container can be a bed or an elongated flexible cloth or perforated membrane holding a substrate. This cloth or membrane can allow gas exchange between the growth container and the growth chamber, and can be made of plastic, textile or a bio-based or biodegradable material. Upon placing the substrate on the bed, the first mesh can be vertically lowered over the substrate. Further, a second mesh can be lowered over the first mesh. Such arrangement facilitates fungal biomass growth from the substrate and through the first and second meshes.

Step 1502 can involve harvesting by moving the second mesh relative to the first mesh causing the fungal biomass to shear. In some embodiments, the moving of the second mesh involves rotating a rotator coupled to the second mesh such that the second mesh translates over the first mesh and moves the second mesh toward the bottom side of the growth container. For example, FIG. 12B illustrates an example of the harvesting by rotating the mesh roller 1222 coupled to the second mesh 1220 causing the fungal biomass 1230 to be sheared off the first mesh 1220 and deposited on the second mesh 1210.

In some embodiments, the second mesh can be pulled (e.g., in a horizontal direction) approximately parallel to the first mesh and the growth container to harvest the fungal biomass over the second mesh, separating the second mesh from the growth container. In some embodiments, the second mesh may be moved in a peeling-like motion, e.g., as shown in FIG. 8A. In some embodiments, a cutter may be disposed between the first mesh and the second mesh for separating the fungal biomass and the second mesh from the first mesh, e.g., as shown in FIGS. 12D and 12E.

Step 1503 can involve automatically transporting the fungal biomass on the second mesh to a delivery area. In some embodiments, the transporting of the harvested fungal biomass can involve providing a platform in the delivery area proximate to the growth container so as to receive the fungal biomass from the second mesh, and providing a stopper on the platform at an end opposite the growth container so as to stop the fungal biomass from advancing off the platform while forming a fungal biomass layer on the platform for easy handling. In some embodiments, the method 1500 can further involve providing a cutter between the growth container and the platform to separate the fungal biomass on the second mesh while the second mesh is moving. In some embodiments, the transporting of the harvested fungal biomass can further involve coupling a lift system or a conveyor to the platform; and conveying, via the lift system or the conveyor, the harvested fungal biomass layer to a specified location. Processes involved in the step 1503 are illustrated in FIGS. 12B and 12C, as an example.

In some embodiments, once the fungal biomass (e.g., mycelium, primordia, mushroom, etc.) is removed from the substrate, the substrate may be discarded. In other embodiments, the substrate can be reused.

According to embodiments described herein, the harvested fungal biomass contains no portions of the inoculated substrate, thereby making the harvested fungal biomass suitable for use as or in food products. In some embodiments, the fungal biomass or portions thereof are shaped, spiced, and cooked and provided as a food product, including but not limited to a meat alternative. In some embodiments, the shaping of the fungal biomass can be achieved by selecting the appropriate shape of a growth container (e.g., cylindrical, conical, rectangular, etc.) or shape in which the fungal biomass is cut.

As used herein, the terms “about” or “approximately” mean±10% of an associated value, unless the context requires otherwise.

### EXAMPLES

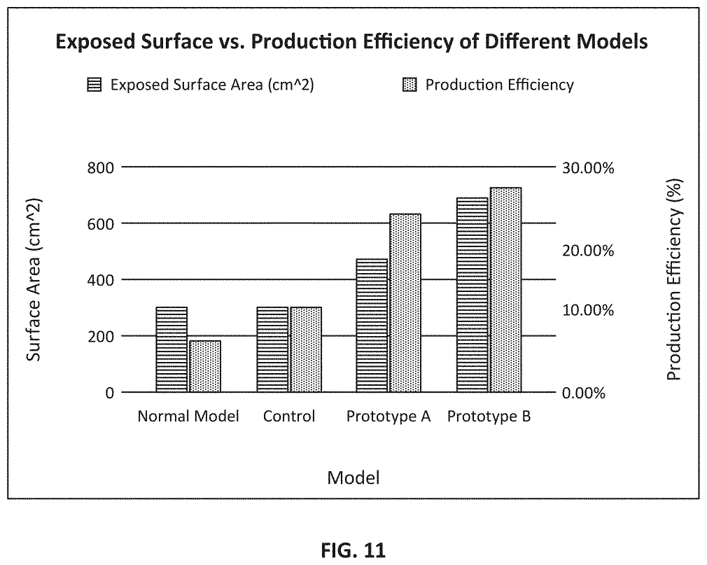

The following trial tests three prototypes of growth containers with varying surface exposure. The reference model is the state-of-the-art tray system, which has a calculated surface open to the environment of the growth chamber of 300 cm². This is the surface allowing gas and moisture exchange during cultivation in the growth chamber. Comparatively, the surface of Prototype A and Prototype B are 471.24 cm², and 687.62 cm² respectively. See FIG. 11.

Prototypes A and B are growth cylinders varying in their length and diameter. Prototype A was 20 cm long and 10 cm wide, while prototype B was 21.5 cm long and 8.5 cm wide. We measured and compared the biological efficiency of these different growth containers after several days of incubation in the growth chamber. The biological efficiency is the ratio of wet fresh mycelium mass to dry substrate mass. The parameters of growth inside the growth chamber were as follows:
Relative humidity between 80% and 100%; Temperature between 20° C. and 28° C.; CO₂ concentration between 2% and 15%; and
Light exposure was varied from total obscurity or different colored lights, in various patterns of ON/OFF periods.

The average biological efficiency achieved by the conventional “normal” model (state-of-the-art tray system) calculated from 43 trays over 3 incubation trials was 6.89%. The specific control model (tray system) placed within the prototype run achieved a biological efficiency of 11.18%. In comparison, Prototype A and B had biological efficiency of 23.62% and 27.22% respectively. FIG. 11.

Prototype B was the most efficient at producing biomass with a 143.47% increase in biological efficiency, compared to the control model, and a 295.07% increase in biological efficiency compared to the average normal model efficiency.

Additionally, these results also suggest that other factors besides substrate composition and environmental conditions should be considered to improve the biological efficiency. When comparing the surface of Prototype A and Prototype B with the standard model, it is observed that prototype A and B have 1.57 times and 2.29 times the exposed surface of the standard model, respectively. This data suggest that there is a much greater capacity for biological efficiency in terms of the substrate packing models conventionally used, by maximizing the open surface to substrate mass ratio.

Although concepts of the present disclosure related to growing and harvesting may be discussed in reference to mycelium in some cases, it does not limit the scope of the present disclosure. For example, growth containers, a growth chamber or environment, tubing systems, harvesting systems and other features herein can be adapted for any other fungal biomass e.g., for primordia, fruiting bodies (e.g., mushrooms), mycelium composites, etc., or a hybrid fungal biomass formed by a combination of one or more of the fungal biomass.

As discussed herein, the terms “substrate” can refer to raw material or growth medium used for growing fungal biomass. The substrate can be organic or non-organic. The substrate can be an inoculated substrate. The substrate can be solid-state, semi-solid. The systems herein can be used with other growth medium for growing and harvesting fungal biomass. For example, growth media can be a medium or a feedstock. The medium can be a substance in which an organism lives or is cultured and can be nutrient-rich. The feedstock can be a renewable, biological material that can be used directly as a fuel, or converted to another form of fuel or energy product.

As discussed herein, a “mesh” of the present disclosure can be a perforated sheet, a metal mesh, a metal wire weave, a fabric with holes, or a plastic perforated membrane made of bio-based or non-bio-based plastic, biodegradable or non-biodegradable plastic. This mesh can be the one coupled to a growth cylinder for holding a substrate or the one spaced from the growth cylinder used for harvesting. In some embodiments, the mesh can be spread over the planar bed. The perforated sheet can be made of aluminum, stainless steel, or other metal or alloys. A sheet thickness can be in a range 0.5 mm to 4 mm. Shape of holes in the mesh can be square, circular, triangular, diamond, or slotted. The mesh has an open area in a range between 15% to 70%. The hole size of the perforated sheet can be in the range between 0.5 mm to 25 mm. The metal wire weave can be made of gauge in a range between 0.2 mm-4 mm. A shape of the weave can be square with an aperture (size of the hole) between 0.025 mm to 50 mm. A plastic perforated membrane can have multiple perforations in the range of 0.01 mm to 25 mm diameter. A fabric mesh can be made of rugged natural, synthetic, and/or composite fibers. In some embodiments, a pulling force on the mesh can be more than 700 kg/m².

Disjunctive language such as the phrase “at least one of X, Y, or Z,” unless specifically stated otherwise, is intended to be understood within the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

All references, including publications, patent applications, and patents, cited herein are hereby incorporated by reference to the same extent as if each reference were individually and specifically indicated to be incorporated by reference and were set forth in its entirety herein.

What is claimed is:

1. A harvesting system comprising:
a growth container for growing fungal biomass, the growth container comprising a first mesh through which the fungal biomass grows;
a second mesh disposed over the first mesh such that the fungal biomass grows further through the second mesh; and
a rotator or a slider coupled to the second mesh and configured to move the second mesh relative to the first mesh causing the fungal biomass to shear and transport the fungal biomass on the second mesh to a delivery area.

2. The harvesting system of claim 1, wherein the rotator is configured to translate the second mesh around and toward the bottom side of the growth container.

3. The harvesting system of claim 1, wherein the slider is configured to pull the second mesh approximately parallel to the growth container and separated from the growth container.

4. The harvesting system of claim 1, wherein the second mesh is configured to peel off with respect to the first mesh to separate the fungal biomass from the growth container.

5. The harvesting system of claim 1, further comprising:
a cutter movably disposed between the first mesh and the second mesh to separate the fungal biomass and the second mesh from the first mesh.

6. The harvesting system of claim 1, further comprises:
a platform located in the delivery area proximate to the growth container so as to receive the fungal biomass from the second mesh; and
a stopper located on the platform at an end opposite the growth container so as to stop the fungal biomass from advancing off the platform while forming a fungal biomass layer on the platform for easy handling.

7. The harvesting system of claim 6, further comprising:
a cutter disposed between the growth container and the platform to separate the fungal biomass on the second mesh while the second mesh is moving.

8. The harvesting system of claim 7, wherein the cutter is an elongated cutter comprising:
a cutting edge oriented against the second mesh; and
a top surface over which the fungal biomass is movable from the second mesh to the platform.

9. The harvesting system of claim 6, wherein the platform is further coupled to a lift system or a conveyor to convey a harvested fungal biomass layer to a specified location.

10. The harvesting system of claim 1, further comprising:
a plurality of growth containers vertically spaced from each other, each growth container being same as the growth container for growing the fungal biomass; and
a plurality of harvesting systems, each harvesting system respectively coupled to one of growth container of the plurality of growth containers, each harvesting system comprising the second mesh relatively slidable with respect to the first mesh of the respective growth container.

11. The harvesting system of claim 10, wherein the plurality of growth containers is supported on columns.

12. The harvesting system of claim 1, wherein the second mesh is made of plastic, aluminum, or steel.

13. The harvesting system of claim 1, wherein the second mesh is coupled to a motor to automatically harvest the fungal biomass.

14. The harvesting system of claim 1, wherein the second mesh is coupled to a motor to automatically move the second mesh, or to a manual slider to manually move the second mesh.

15. A method for harvesting fungal biomass, the method comprising:
providing a growth system for growing the fungal biomass, the growth system: a growth container holding an inoculated substrate; a first mesh coupled to the growth container and extending over the inoculated substrate such that the fungal biomass grows through the first mesh; and a second mesh disposed over the first mesh such that the fungal biomass grows further through the second mesh;
harvesting by moving the second mesh relative to the first mesh causing the fungal biomass to shear; and
automatically transporting the fungal biomass on the second mesh to a delivery area.

16. The method of claim 15, the moving of the second mesh comprises:
rotating a rotator coupled to the second mesh such that the second mesh translates over the first mesh and moves the second mesh toward the bottom side of the growth container.

17. The method of claim 15, the moving of the second mesh comprises:
pulling the second mesh approximately parallel to the first mesh and the growth container to harvest the fungal biomass over the second mesh; and
separating the second mesh from the growth container.

18. The method of claim 15, wherein the transporting of the harvested fungal biomass comprises:
providing a platform in the delivery area proximate to the growth container so as to receive the fungal biomass from the second mesh; and
providing a stopper on the platform at an end opposite the growth container so as to stop the fungal biomass from advancing off the platform while forming a fungal biomass layer on the platform for easy handling.

19. The method of claim 18, further comprising:
providing a cutter between the growth container and the platform to separate the fungal biomass on the second mesh while the second mesh is moving.

20. The method of claim 19, wherein the transporting of the harvested fungal biomass further comprises:
coupling a lift system or a conveyor to the platform; and
conveying, via the lift system or the conveyor, the harvested fungal biomass layer to a specified location.
